(19) United States
(12) Patent Application Publication
(10) Pub. No.: US 2015/0010973 A1
(43) Pub. Date: Jan. 8, 2015

(54) FUNGAL STRAINS WITH GENETIC MODIFICATION RELATING TO A CARBOXYLIC ACID TRANSPORTER

(71) Applicants: Andreas Aurich, Leipzig (DE); Martina Holz, Dresden (DE); Anne Kretzschmar, Troisdorf (DE); Christina Otto, Dresden (DE); Gerold Barth, Dresden (DE); Isabel Waengler, Berlin (DE); Roland Arno Müller, Markranstadt (DE)

(72) Inventors: Andreas Aurich, Leipzig (DE); Martina Holz, Dresden (DE); Anne Kretzschmar, Troisdorf (DE); Christina Otto, Dresden (DE); Gerold Barth, Dresden (DE); Isabel Waengler, Berlin (DE); Roland Arno Müller, Markranstadt (DE)

(73) Assignee: THYSSENKRUPP INDUSTRIAL SOLUTIONS AG, Essen (DE)

(21) Appl. No.: 14/364,969

(22) PCT Filed: Dec. 12, 2012
(86) PCT No.: PCT/EP2012/075268

§ 371 Date: Jun. 12, 2014

(30) Foreign Application Priority Data
Dec. 12, 2011 (DE) 10 2011 056 290.7

(51) Int. Cl. C12N 15/81; C12P 7/50; C12P 7/46; C12P 7/40

(52) U.S. Cl. CPC C12N 15/815; C12P 7/40; C12P 7/50; C12P 7/46
USPC 435/143; 435/254.2; 435/254.21; 435/254.23; 435/254.22; 435/254.11; 435/254.3; 435/254.5; 435/254.9; 435/256.7; 435/471; 435/136; 435/145

(57) ABSTRACT

The invention relates to fungal strains having at least one genetic modification which leads to a reduction of the activity of at least one fungal carboxylic acid transporter and to a method for producing or using said fungal strains.

The present invention relates to fungal strains with genetic modification relating to a carboxylic acid transporter, and methods for the production or use of such fungi.

### FIELD OF THE INVENTION

Interest in biotechnologically produced carboxylic acids, for example of the citrate cycle, as a potential intermediate starting material and as an alternative to petrochemical production processes in industry is steadily increasing. Many of these carboxylic acids have the potential to function as “building block” chemicals. Important precursors for chemical synthesis, polyester production and other processes can be formed from said carboxylic acids. Thus, for example, the succinate important for industrial purposes is at present still mainly obtained petrochemically from maleic anhydride starting from butane. The petrochemical production of succinate using heavy metal catalysts, organic solvents, high temperatures and pressures is costly.

The market for succinate was therefore relatively small up to now. On account of increasing oil prices, but also because of the continuing need for synthetic products, the biotechnological production of carboxylic acids, in particular of succinate, and the development of appropriate processes is moving increasingly strongly into the focus of the production industry.

Carboxylic acids such as succinate are formed naturally by many microorganisms, plants and animals as an intermediate in the central metabolism or as a metabolic end product. The majority of the natural and optimized producers are bacterial strains, which, for example, accumulate succinate, especially under anaerobic conditions. On account of the disadvantages of the bacterial production processes, such as, for example, the need for complex culture media, the potential pathogenicity and particularly the low acid tolerance, microbial fungi, however, move more and more strongly into the focus of the investigations on the development of processes for the production of organic acids such as succinate.

Various genetic modifications are discussed, in particular to influence succinate formation in fungi. Thus, various genes of the citrate cycle were disrupted, it being shown that the deletion of to SDH1, which codes for a subunit of SDH (succinate dehydrogenase), led to a 1.6-fold increase in succinate production, in combination with a FUM1 deletion (gene that codes for fumarase) even to a 2.6-fold increase in succinate production (Arikawa et al. 1999b).

The disruption of the genes of SDH, which consists in S. cerevisiae of at least four subunits (Ghezzi et al. 2009), in Said strains of the genus Saccharomyces resulted in a reduction of the SDH activity and an increase in succinate formation, a maximum of 0.3 g L⁻¹ of succinate being formed (Kubo et al. 2000).

With the aid of these findings, S. cerevisiae strains were later constructed with the aim of succinate production for industrial use (Raab and Lang 2011). With simultaneous deletion of SDH1, SDH2, IDH1 (the gene for isocitrate dehydrogenase), and IDP1 (which codes for a mitochondrial isoenzyme of IDH), a maximum of 3.62 g L⁻¹ of succinate were produced after 168 h. However, ethanol, glycerol, acetate, α-ketoglutarate (AKG) and pyruvate were also formed as by-products.

Further changes, such as the expression of IDH1 and SDH2 or IDH1 and DIC1 controlled by a repressible promoter, which codes for a dicarboxylate transporter for the transport of succinate and malate from the cytosol into the mitochondria, were described in the literature (Raab and Lang 2011).

In the course of this, it was attempted to deregulate the expression of further genes (PYC1, ACS1, CIT1, ACO1, ICL1, MLS1, MDH2 and CIT2) by means of control by a constitutive promoter (pADH1—promoter of alcohol dehydrogenase) or to replace it by genes of Craptree-negative yeasts (Raab and Lang 2011).

US20110104771 discloses that an S. cerevisiae strain with an ICL gene from K. lactis, the malate synthase gene from S. cerevisiae, with a gene for the PEPCK from M. succiniciproducens, the gene MDH2 from S. cerevisiae and with genes for fumarase (from Rhizopus oryzae) and fumarate reductase (from Trypanosoma brucei) can produce up to 6.3 g L⁻¹ of succinate after 7 days.

### SUMMARY OF THE INVENTION

An object of the invention consists in making available processes or organisms, with the aid of which the production of organic acids can be accomplished economically.

A further object of the invention consists in making available processes or organisms for the production of organic acids, which have advantages compared to the bacterial processes.

### DETAILED DESCRIPTION OF THE INVENTION

These objects are achieved by the features of the independent claims. The subclaims indicate preferred embodiments.

Accordingly, a fungal strain is provided, having a genetic modification which leads to a decrease in the activity at least of one fungus-specific carboxylic acid transporter.

Said carboxylic acid transporter is a membrane transport protein, which is able to transport carboxylic acids through the cell membrane and optionally the cell wall. Preferably, in this connection it is a transporter for dicarboxylic acids, preferably a transporter for carboxylic acids of the citrate cycle or adjacent metabolic pathways. These can be symporters (co-transporters), but also uniporters or antiporters, very particularly preferably transporters for at least one organic acid selected from the group containing fumarate, lactate, pyruvate, malate, malonate and/or succinate.

For Kluyveromyces lactis and Schizosaccharomyces pombe, dicarboxylate permeases have already been described which mediate the transport of succinate/malate/fumarate (KlJen2p) or lactate/pyruvate (KLJen1p) as well as the transport of succinate/malate/malonate (SpMae1p). The two first-mentioned belong to the group consisting of the JEN transporters, while the last-mentioned belong to the group consisting of the MAE transporters.

The term fungus-specific, as used here, is intended to make clear that said carboxylic acid transporter is encoded in the genome of the fungus. Alternatively, the term homologous can also be used. Particularly preferably, it is intended that said fungus has increased extracellular carboxylic acid production.

The term carboxylic acids as used here is identical to the term organic acids and relates to all those metabolic products of said fungus which can be designated as carboxylic acids. Preferably, the term designates the carboxylic acids of the citrate cycle and adjacent metabolic pathways, in particular pyruvate, α-ketoglutarate, malate, oxalacetate, citrate, isocitrate, fumarate, malonate, lactate and/or succinate. Most of these acids are dicarboxylic acids.

Likewise, a method for the genetic modification of a fungal strain for the purpose of increasing the extracellular carboxylic acid production is provided, said method leading to a decrease in the activity at least of one fungus-specific carboxylic acid transporter.

Preferably, it is provided here that the decrease in the activity of the carboxylic acid transporter is achieved by at least a property or a step selected from the group comprising:
a) inhibition or reduction of the expression of a gene coding for a carboxylic acid transporter
b) expression of a dysfunctional or decreased activity carboxylic acid transporter, and/or
c) inhibition or reduction of the activity of the expressed carboxylic acid transporter
   is or will be brought about.

The inhibition or reduction of the expression can take place, for example, by inhibition or reduction of the transcription of the coding gene or the translation of the generated mRNA. The expression of a dysfunctional or decreased activity carboxylic acid transporter can be brought about, for example, by deletion, insertion, substitution or point mutation in the coding gene.

To this end, for example, the following methods are suitable: In gene silencing, the gene regulation is carried out by an inhibition of the transfer (transcription) of a piece of genetic information from the DNA to the mRNA (transcriptional gene silencing) or the subsequent transmission (translation) of the information stored on the mRNA to a protein (post-transcriptional gene silencing). Transcriptional gene silencing is a result of epigenetic alterations of the DNA, such as, in particular, DNA methylation or histone modifications. By means of these modifications of the histone ends, a type of heterochromatic state around the gene is created, which bars it from binding to the transcription machine (RNA polymerase, transcription factors, etc.). The classical example is the phenomenon designated as position effect variegation (PEV). It changes the chromatin state and thus controls the transcription activity of the gene or the gene region concerned. Post-transcriptional gene silencing (PTGS) is designated as the processes of gene silencing which take place only after the transcription of the genetic information from the DNA to the transferring mRNA. The forms of post-transcriptional gene silencing include, in particular, nonsense-mediated mRNA decay (NMD) and RNA interference (RNAi). While nonsense-mediated mRNA decay serves primarily for the avoidance of nonsense point mutations, RNA interference is a mainly regulatory process involving specific RNA molecules, such as miRNA and siRNA. Post-transcriptional gene silencing can lead to an intensified degradation of the mRNA of a certain gene. Owing to the degradation of the mRNA, the translation and thus the formation of the specific gene product (usually a protein) is prevented. Moreover, a gene-specific direct inhibition of the translation is possible as a consequence of the post-transcriptional gene silencing. Gene knockout is understood as meaning the complete switching off of a gene in the genome of an organism. The switching off of the gene is achieved by gene targeting. A deletion, also gene deletion, in genetics is a variant of the gene mutation or in which a nucleotide sequence or a part up to the entire chromosome is missing. A deletion is therefore always a loss of genetic material. Any number of nucleobases can be deleted, from an individual base (point mutation) up to the chromosome. There is a difference between interstitial and terminal deletion. The former describes a loss within the chromosome, the latter a losing of an end section that is a part of the telomer region.

As a result of deletion, an erroneous protein can result from the mRNA created from the DNA after translation. A reading frame shift mutation can be produced by the deletion of base pairs if a number of base pairs was removed that is not divisible by three. In the case of site-specific or selective mutagenesis, a selective modification of the DNA is made possible with the aid of a recombinant DNA. Thus individual nucleobases of a gene can be exchanged selectively or else whole genes can be removed. Alternatively, by means of a deletion cassette that has been integrated into a vector, a deletion in the gene to be mutated can be produced.

Alternatively, the inhibition or reduction of the expression of a gene can take place by cloning of a heterologous promoter, which is inhibitable by an exogenous factor, for example a tetracycline-regulated tetO promoter. Likewise, the inhibition or reduction of the expression of a gene can take place by cloning of a heterologous promoter, which is weaker than that of the homologous promoter concerned, for example pPOT1.

The inhibition or reduction of the activity of the expressed carboxylic acid transporter can take place, for example, by transfer of inhibitors or synchronous expression of suitable, heterologous inhibitors.

Said fungal strain is preferably a strain of microbial fungi.

The term microbial fungus, as used here, should in particular comprise those fungi which can be cultured using biotechnological methods and are suitable in particular for fermentative production methods, for example in suspension cultures.

Preferably, it is intended that the fungus is a member of the group consisting of the ascomycetes (Ascomycota). Particularly preferably, it is a member here of the group consisting of the hemiascomycetes or of the euascomycetes.

In a furthermore preferred embodiment, said fungal strain is a yeast or a mould fungus. Said fungus particularly preferably belongs to a genus that is selected from the group containing Saccharomyces; Schizosaccharomyces; Wickerhamia; Debayomyces; Hansenula; Hanseniospora; Pichia; Kloeckera; Candida; Zygosaccharomyces; Ogataea; Kuraishia; Komagataella (Pichia); Yarrowia; Metschnikowia; Williopsis; Nakazawaea; Kluyveromyces; Cryptococcus; Torulaspora; Torulopsis; Bullera; Rhodotorula; Sporobolomyces; Pseudozyma; Saccharomycopsis; Saccharomycodes; Aspergillus; Penicillium; Rhizopus; Trichosporon and/or Trichoderma.

In a furthermore preferred embodiment, it is provided that the at least one carboxylic acid transporter originates from the group consisting of the JEN transporters and/or the MAE transporters.

For Kluyveromyces lactis and Schizosaccharomyces pombe, dicarboxylate permeases have already been described which mediate the transport of succinate/malate/fumarate (KlJen2p) or lactate/pyruvate (KLJen1p) as well as the transport of succinate/malate/malonate (SpMae1p). The two first-mentioned belong to the group consisting of the JEN transporters, while the last-mentioned belong to the group consisting of the MAE transporters.

Until now, 35 proteins, which were encoded by putative orthologs of the K. lactis genes KlJEN1 and KlJEN2, have been found in 17 different fungal species (13 hemiascomycetes, 7 euascomycetes). Here, the number of JEN orthologs varies from one (e.g. S. cerevisiae) up to 6 orthologous genes in Y. lipolytica. The following table gives an overview of the group consisting of the JEN transporters, and also, in the last lines, of the known MAE transporters:

Known carboxylic acid transporters from the group consisting
of the JEN or MAE transporters in microbial fungal strains.

| | | | | | Length | Number of trans- |
|---|---|---|---|---|---|---|
| Type | Species | Working name | EMBL | Swiss Prot | (aa) | membrane domains |
| JEN | K. lactis | KLLA-E-KLJEN1 | AJ585426 | Q70DJ7 | 600 | 12 |
| | | KLLA-F-KLJEN2 | AJ627630 | Q701Q9 | 528 | 11 |
| | Sac. cerevisiae | SACE-K-JEN1 | U24155 | P36035 | 616 | 12 |
| | Sac. paradoxus | SAPA-X1-J101 | | | 616 | 12 |
| | Sac. mikatae | SAMI-X1-J101 | | | 616 | 12 |
| | Sac. kudriavzevii | SAKU-X1-J101 | | | 614 | 10 |
| | Sac. bayanus | SABA-X1-J101 | | | 615 | 12 |
| | K. thermotolerans | KLTH-G-J201 | | | 542 | 11 |
| | K. waltii | KLWA-X1-J201 | | | 534 | 11 |
| | Sac. kluyveri | SAKL-X1-J101 | | | 598 | 10 |
| | | SAKL-X2-J201 | | | 523 | 11 |
| | | SAKL-X3-J001 | | | 531 | 11 |
| | A. gossypii | ASGO-A-J001 | AAS50559 | Q75E88 | 500 | 10 |
| | | ASGO-B-J201 | AAS50561 | Q75E76 | 478 | 9 |
| | | ASGO-F-J101 | AAS53704 | Q753H9 | 500 | 9 |
| | D. hansenii | DEHA-D-J202 | CAG87482 | Q6BR62 | 511 | 10 |
| | | DEHA-F-J101 | CAG89500 | Q6BL03 | 513 | 12 |
| | | DEHA-F-J201 | CAG89946 | Q6BJV3 | 506 | 12 |
| | C. albicans | CAAL-D-J001 | EAK98054 | Q5A5U2 | 513 | 10 |
| | | CAAL-C-J101 | EAK97104 | Q5A2W4 | 541 | 10 |
| | Y. lipolytica | YALI-B-J101 | CAG83351 | Q6CE14 | 529 | 10 |
| | | YALI-C-J102 | CAG82184 | Q6CBU1 | 499 | 10 |
| | | YALI-C-J201 | CAG82410 | Q6CB72 | 523 | 10 |
| | | YALI-D-J204 | CAG81250 | Q6C8F4 | 502 | 10 |
| | | YALI-D-J202 | CAG81440 | Q6C7X3 | 503 | 10 |
| | | YALI-E-J203 | CAG80310 | Q6C3Q6 | 524 | 10 |
| | N. crassa | NECR-X1-J201 | EAA33605 | Q7SB47 | 557 | 11 |
| | | NECR-X2-J202 | EAA32361 | Q9P732 | 518 | 9 |
| | A. fumigatus | ASFU-G-J202 | EAL86916 | Q4WGM5 | 501 | 11 |
| | | ASFU-H-J001 | EAL85090 | Q4WB22 | 520 | 10 |
| | | ASFU-B-J201 | EAL93241 | Q4X1M4 | 511 | 9 |
| | A. nidulans | ASNI-A-J201 | | | 495 | 10 |
| | | ASNI-A-J202 | | | 514 | 11 |
| | A. oryzae | ASOR-A-J201 | | | 508 | 11 |
| | | ASOR-G-J202 | | | 512 | 10 |
| MAE | S. pombe | n/a | n/a | P50537 | 438 | 10 |
| | Y. lipolytica | n/a | n/a | n/a | | |

A = Aspergillus; Sac = Saccharomyces; N = Neurospora; Y = Yarrowia; C = Candida; D = Debaromyces and K = Kluyveromyces

Preferably, it is provided here that the at least one carboxylic acid transporter originates from the group consisting of the JEN transporters and/or the MAE transporters listed in the following table.

| | Belonging to the | | | Deletion | |
|---|---|---|---|---|---|
| | dicarboxylate | Working | Alias | cassette | Vector |
| Organism | permease group: | name | name | SEQ ID No | SEQ ID No |
| Y. lipolytica | KIJEN1 | YALI-B- | JEN2 | 1 | 2 |
| | | J101 | | | |
| | KIJEN1 | YALI-C- | JEN1 | 3 | 4 |
| | | J102 | | | |
| | KIJEN2 | YALI-C- | JEN6 | 5 | 6 |
| | | J201 | | | |
| | KIJEN2 | YALI-D- | JEN4 | 7 | 8 |
| | | J204 | | | |
| | KIJEN2 | YALI-D- | JEN3 | 9 | 10 |
| | | J202 | | | |
| | KIJEN2 | YALI-E- | JEN5 | 11 | 12 |
| | | J203 | | | |
| Schizosaccharomyces | SpMae1 | | SPMAE1 | 13 | 14 |
| pombe | | | | | |
| Y. lipolytica | SpMae1 | | MAE1 | 13 | 14 |

Experimentally, the gene concerned was deleted by means of a deletion cassette integrated into a vector (SEQ ID Nos 1, 3, 5, 7, 9 and 11). The vector used is in each case pUCBM21, which was originally developed by Boehringer (SEQ ID Nos 2, 4, 6, 8, 10, 12). However, all other suitable vectors known from the prior art can be used.

Particularly preferably, it is provided that the at least one carboxylic acid transporter is encoded by the gene YALI-D-J204 (EMBL-ID: CAG81250; Swiss-Prot ID: Q6C8F4). Said gene is always also designated in the context of this application as “JEN4”. Likewise, it is particularly preferably provided that said fungus belongs to the genus Yarrowia, particularly preferably to the species Yarrowia lipolytica.

As shown above, the Y. lipolytica has altogether 6 JEN orthologs. This high number of genes for putative carboxylate transporters reflects the great potential of the yeast Y. lipolytica for the production of organic acids, such as, for example, succinate, malate or fumarate. Moreover, Y. lipolytica is extensively characterized genetically and physiologically, can make use of a broad spectrum of substrates and is tolerant to low pHs.

Further, it is preferably provided that said fungus has at least one further genetic modification selected from the group comprising:
reduction of the activity or expression of succinate dehydrogenase (SDH), for example by replacement of the native promoter of SDH2 by a weak promoter, (in the case of glucose and glycerol as a substrate pPOT1, for example, is suitable), or by use of a suitable deletion cassette,
increase in the activity or expression of pyruvate carboxylase (PYC), for example by overexpression of the corresponding gene PYC1, and/or
increase in the activity or expression of isocitrate lyase (ICL), for example by overexpression of the corresponding gene ICL1.

All these genetic modifications are connected with the formation of carboxylic acids in the context of the citrate cycle. The modifications mentioned are described in detail in the methods section.

Further suitable genetic modifications, which can be used in combination with the modifications mentioned, concern a reduction of the activity or expression of the genes SDH1, SDH3 and/or SDH4, the introduction of a heterologous gene selected from the group coding for phosphoenolpyruvate carboxykinase (PCK1), fumarate reductase and/or fumarase (FUM1), and/or the reduction of the activity or expression of the gene coding for isocitrate dehydrogenase (IDH1) as well as at least one of the genes coding for the mitochondrial dicarboxylate carrier (DIC1) and/or SDH2, and optionally at least one of the genes FUM1, osmotic growth protein (OSM1), malate dehydrogenase (MDH3) and/or citrate synthase (CIT2). The reduction of the expression can take place, for example, by means of a heterologous promoter, which is inhibitable by an exogenous factor, preferably a tetracycline-regulated tetO promoter. Alternative methods (gene silencing, gene knock out, deletion) are shown further above.

The genes mentioned are identified in the following table, as an example the UniProt entries for Saccharomyces cerevisiae were chosen here. It is understood that the person skilled in the art with the aid of these details can find the corresponding genes in other fungal strains, in particular in Yarrowia, without difficulty.

| Name | Abbreviation | UniProt |
|---|---|---|
| Succinate dehydrogenase complex, subunit | SDH2 or SDHB | P21801 |
| Succinate dehydrogenase complex, subunit | SDH1 or SDHA | Q00711 |
| Succinate dehydrogenase complex, subunit b | SDH3 or CYB3 | P33421 |
| Succinate dehydrogenase complex, small | SDH4 or ACN18 | P37298 |
| subunit b | | |
| Pyruvate carboxylase | PYC1 | P11154 |
| Isocitrate lyase | ICL1 | P28240 |
| Phosphoenolpyruvate carboxykinase | PCK1 | P10963 |
| Fumarate reductase, subunit A | | |
| Fumarate reductase, subunit C | | |
| Fumarate reductase, subunit D | | |
| Fumarase | FUM1 | P08417 |
| Isocitrate dehydrogenase | IDH1 | P28834 |
| Mitochondrial dicarboxylate carrier | DIC1 | Q06143 |
| Osmotic growth protein | OSM1 | P21375 |
| Malate dehydrogenase | MDH3 | P32419 |
| Citrate synthase | CIT2 | P08679 |

In a further embodiment of the invention, a method for the production of carboxylic acids is provided, a fungal strain as claimed in one of the previous claims being used in the method.

Particularly preferably, it is provided here that hexoses, such as, for example, glucose or sucrose, or alcohols, such as, for example, glycerol, are used as a carbon source. Glucose and sucrose are particularly suitable as they are inexpensive substrates.

Glycerol is obtained in large amounts as a waste product, for example, in the production of biodiesel and is therefore likewise an inexpensive substrate. For glycerol, it can moreover be said that its uptake by the fungi is not affected by an impairment of a carboxylic acid transporter.

Further, it is preferably intended that the fungal strains are cultured in a medium, and where furthermore the oxygen uptake rate (OUR) is regulated in a range between ≧5 and ≦50 mmol O₂/l*h.

In this range, such have investigations of the Applicant revealed, the highest production rates are achieved. Preferably, the oxygen uptake rate is regulated in the range between ≧10 and ≦40 mmol O₂/l*h. Particularly preferably, the oxygen uptake rate is regulated in a range between ≧20 and ≦30 mmol O₂/l*h. Extremely preferably, the oxygen uptake rate is regulated in a range between 25±3 mmol O₂/l*h.

Technically, in a fermenter the oxygen entry OTR can be influenced by the air gassing rate (1 min⁻¹), the stirrer speed (U min⁻¹), the pressurization of the fermenter and the gas composition (proportion of O₂ in the gas mixture). The oxygen uptake rate OUR can be determined based on the known introduced gas composition as well as the amount of gas and the measurement of the oxygen concentration in the waste gas with the aid of generally known equations.

### BRIEF DESCRIPTION OF THE FIGURES

The figures show:

FIG. 1 Schematic representation of the expression cassette for the exchange of the SDH2 promoter for the promoter of the genes ICL1, GPR1 or POT1. All expression cassettes contained URA3 as a selection marker, flanked by the lox sites and homologous regions of the SDH2 promoter and SDH2-ORF. The SDH2-ORF was directly fused with the respective promoter. The pICL1-containing expression cassette had a size of 4.9 kb, the pPOT1 cassette 3.8 kb and the GPR1B cassette was 4.2 kb in size.

FIG. 2 Plasmids pSpIvS-Ura, pSpPS-Ura and pSpGS-Ura with the expression cassettes for the exchange of the SDH2 promoter for pICL1, pPOT1 or GPR1B. The marker gene URA3 flanked by the lox sites is contained as a selection marker.

FIG. 3 Vector construction of the plasmid p64PIC starting from p64PYC1 and p64ICL1. In addition to the desired genes with their own promoter and terminator region, p64PIC contained the URA3 allele ura3d4 as a multicopy marker gene and rDNA as an integration sequence.

FIG. 4 PYC and ICL activities of the wild-type strain Y. lipolytica H222 and the transformants H222-AZ8 T3, T4, T5 and H222-AZ9 T2 and T3 in MG medium. The strains were cultured in 100 ml each of MG medium (growth medium) in 500 ml Erlenmeyer flasks at 28° C. and 220 rpm. Sampling for the activity measurement took place after 4 h.

FIG. 5 Schematic representation for the deletion of JEN4 by homologous recombination in the promoter and terminator region using the URA3 contained deletion cassette.

FIG. 6 Growth (a) and succinate production (b) of the wild-type strain Y. lipolytica H222 and the transformants H222-AZ7 T11 and T23 in YNB medium. The strains were cultured with 5% glycerol in 150 ml each of culture medium in 500 ml Erlenmeyer flasks at 28° C. and 220 rpm. The succinate contents in the culture supernatant were determined by ion chromatography.

FIG. 7 Strategy for the construction of the strain H222-AZ10.

FIG. 8 PYC activities of the wild-type strain Y. lipolytica H222 and the transformants H222-AZ10 T1, T5 and T9 in MG medium. The strains were cultured in 100 ml each of MG medium (growth medium) in 500 ml Erlenmeyer flasks at 28° C. and 220 rpm. Sampling for the activity measurement took place after 4 h.

FIG. 9 Growth (a) and succinate production (b) of the wild-type strain Y. lipolytica H222 and the transformants H222-AZ7 T11, H222-AZ8 T3 as well as H222-AZ10 T1, T5 and T9 in YNB medium. The strains were cultured with 5% glycerol in 150 ml each of culture medium in 500 ml Erlenmeyer flasks at 28° C. and 220 rpm. The succinate contents in the culture supernatant were determined by ion chromatography.

FIG. 10 Maximally fanned product amounts of the organic acids succinate, malate, α-ketoglutarate (AKG) and fumarate on culturing the strains Y. lipolytica H222, H222-AZ8 T3, H222-AZ7 T11 and H222-AZ10 T1, T5 and T9 in YNB medium. Culturing in YNB medium with 5% glycerol as the C source.

FIG. 11 Percentage proportion of the organic acids succinate (SA), malate (MA), α-ketoglutarate (AKG) and fumarate (FA) in the total acid product on culturing the strains Y. lipolytica H222, H222-AZ8 T3, H222-AZ7 T11 and H222-AZ10 T1, T5 and T9 in YNB medium. Culturing in YNB medium with 5% glycerol as the C source.

FIG. 12 Maximal acid production of the MAE1 deleted transformants. The averaged, maximal acid amount of the H222-SW2ΔMAE1 transformants TF3, TF7 and TF8, as well as of the reference strain H222 from 3-fold culturing in production medium according to Tabuchi et al. (1981) was shown. AKG=α-ketoglutarate, FU=fumarate, MA=malate, SUC=succinate.

FIG. 13 Vector construction of the plasmid p64PYC1 starting from p64T.

FIG. 14 Southern Blot analysis for the confirmation of multiple integration of p64PYC1 in Y. lipolytica H222-S4. The genomic DNA was completely digested using the restrictase EcoRV. For the detection of PYC1, a specific probe was used for the PYC1-ORF (2.1 kb), which was obtained by means of PCR and the primers PYC_ORF_for and PYC_ORF_rev. The black arrow identifies the 8.9 kb sized genomic (g) PYC1 fragment and the red arrow shows the 6.2 kb vectorial (v) PVC1 fragment. λ Molecular weight standard; 1—recipient strain H222-S4; 2—transformant H222-AK1-1; 3—transformant H222-AK1-2; 4—transformant H222-AK1-3; 5—transformant H222-AK1-4; 6—transformant H222-AK1-5; 7—transformant H222-AK1-6; 8—transformant H222-AK1-7.

FIG. 15 Kinetics of the specific pyruvate carboxylase activity of the transformants using a multiple integration of the PYC1 expression cassette in comparison to the wild-type H222. The specific activities were determined during the culturing in 100 ml of minimal medium containing 1% glucose. The characteristic course of the specific activities resulted from the enzyme activity determinations of three independent culturings.

FIG. 16 Growth (a) and succinate production (b) of the strains Y. lipolytica H222 (wild-type) and H222-AM3 in Tabuchi medium. For the assessment of the growth, the optical density of the culture was determined at 600 nm at certain points in time. The strains were cultured with 10% glycerol in 100 ml each of production medium according to Tabuchi et al. (1981) in 500 ml Erlenmeyer flasks at 28° C. and 220 rpm. The determination of the succinate contents was carried out by ion chromatography. The course of the optical densities (OD₆₀₀) and also the succinate production in each case of one of the experiments of a number of repetitions are shown.

### COMPOSITION OF THE CULTURE MEDIUM

### 1. YNB Medium

TABLE 1a
Composition of the YNB culture medium

| | | |
|---|---|---|
| NH₄Cl | 10.0 | g L⁻¹ |
| KH₂PO₄ | 1.0 | g L⁻¹ |
| MgSO₄ × 7 H20 | 1.0 | g L⁻¹ |
| Na₂HPO₄ × 2 H2O | 1.3 | g L⁻¹ |
| CaCl₂ × 6 H₂O | 0.2 | g L⁻¹ |
| NaCl | 0.1 | g L⁻¹ |
| Pharmaceutical glycerol (99%) | 50 | g L⁻¹ |
| Thiamine | 1.0 | mg L⁻¹ |
| FeSO₄ × 7 H₂O | 35 | mg L⁻¹ |
| Trace elements | 1.0 | ml L⁻¹ |
| Dist. water (sterile) | | |

2. Trace elements YNB

| | |
|---|---|
| H₃BO₃ | 0.5 g L⁻¹ |
| CuSO₄ × 5 H₂O | 0.63 g L⁻¹ |
| KI | 0.1 g L⁻¹ |
| MnSO₄ × H₂O | 0.45 g L⁻¹ |
| Na₂MoO₄ × 2 H₂O | 0.24 g L⁻¹ |
| ZnSO₄ × 7 H₂O | 0.71 g L⁻¹ |

3. Tabuchi medium

TABLE 1b
Composition of the medium modified according to Tabuchiet al. (1981):

| | |
|---|---|
| NH₄Cl | 3.0 g L⁻¹ |
| KH₂PO₄ | 0.1 g L⁻¹ |
| MgSO₄ × 7 H₂O | 0.5 g L⁻¹ |
| FeSO₄ × 7 H₂O | 0.035 g L⁻¹ |
| Yeast extract | 2.5 g L⁻¹ |

At the start of the studies, the Y. lipolytica wild-type strain H222 was selected for the investigation of succinate production. H222 had already proved to be a very good acid producer compared to other Y. lipolytica strains with respect to citrate or AKG production. H222 was cultured in a culture medium that was optimized for itaconic acid production by Candida strains.

Glucose (10%), glycerol (10%) and sunflower oil (5%) were selected as C sources. Culturing was carried out in 500 ml Erlenmeyer flasks without baffles in 100 ml of culture medium at 28° C. and 220 rpm. When using sunflower oil as the C source, the liquid cultures were shaken in 500 ml Erlenmeyer flasks with baffles. Preculture was carried out in 50 ml of the same medium and it was incubated for 2-3 days at 28° C. and 220 rpm. The preculture was harvested by centrifugation (3.500 rpm, 5 min, RT) and taken up in production medium without a C source, yeast extract and iron sulfate. After determination of the optical densities, the main cultures were inoculated using 100 ml each of the production medium having an optical density of 2 OD ml⁻¹. Subsequently, culturing took place at 28° C. and 220 rpm for up to 500 h. The analysis of the concentrations of the organic acids secreted during the culturing of Y. lipolytica took place by means of ion chromatography using the ion chromatography system ICS-2100 of Dionex (Sunnydale, USA).

The ion chromatography systems contained the following components: Isocratic pump and conductivity detector IC20, eluent generator EG40, autosampler AS40/autosampler AS50, self-regenerating suppressor ASRS, IonPac AS19 separating column (2×250 mm) with IonPac AS19 precolumn (2×50 mm), Chromeleon Version 6.8 analysis software. The separation parameters of the separating method are listed in Table 2. Preparation of the samples for ion chromatography: At certain times of the culturing, 1 ml each of the culture was removed and prepared depending on the C source contained. Apart from the cultures in which sunflower oil was used as the C source, the samples were centrifuged at maximum speed (5 min, 4° C.) and the supernatant was employed with appropriate dilution in double-distilled water for ion chromatography. If sunflower oil was used as the C source, this had to be removed before the analysis. For this, the sample was treated with 0.5 ml of n-hexane, shaken vigorously for 5 min and subsequently centrifuged for 5 min at maximum speed and 4° C. The aqueous lower phase resulting here was transferred to a new reaction vessel and treated again with 0.3 ml of n-hexane, shaken and centrifuged again. The aqueous phase was removed again and employed with appropriate dilution for the ion-chromatographic analysis. As the culture medium used here was not hitherto optimized for succinate production and the culturing conditions, such as, for example, pH and pO₂, cannot permanently be kept stable on the shaker flask scale, in this and in all following culturing experiments it was dispensed with the calculation of maximum productivities or maximum specific product formation rates.

TABLE 2
Ion chromatography separation parameters and
retention times for the organic acids.

| Column | IonPac AS19 | |
|---|---|---|
| Flow rate | 0.3 ml min⁻¹ | |
| Injection volume | 10 μl | |
| Mobile Phase | dd H₂O | |
| Eluent | KOH | |
| | 0-3 min: isocratic, 5 mM | |
| | 3-25 min: linear to 38 mM | |
| | 25-36 min: isocratic 38 mM | |
| | 36-38 min: linear to 5 mM | |
| | 38-40 min: isocratic, 5 mM | |
| Retention times (min) | Pyruvate | 7.5 min |
| | Malate | 19.6 min |
| | Succinate | 20.3 min |
| | α-Ketoglutarate | 23.4 min |
| | Fumarate | 27 4 min |

### 1. Starting Organisms

For a further molecular biological processing of this strain, an introduction of auxotrophies/selection markers was necessary. The uracil-auxotrophic strain H222-S4 was already present at the start of the studies (Mauersberger et al. 2001). Further auxotrophic strains were constructed. Within this project, only one further uracil-auxotrophic strain (H222-SW2) was employed. H222-SW2 was produced by deletion of the Ku70 gene, the gene product of which plays a role in DNA recombination, in H222-S4.

Expression cassettes were constructed, which in addition to the selection marker URA3 contained the corresponding promoter (pICL1, GPR1B or pPOT1) fused to the start of the SDH2-ORF as well as a homologous region of pSDH2 (FIG. 1). The selection marker URA3 was flanked by the so-called lox sites, whereby a possible later removal of this marker by means of Crelox system was made possible. The homologous regions of pSDH2 and SDH2-ORF are intended to serve for the homologous integration of this expression cassette.

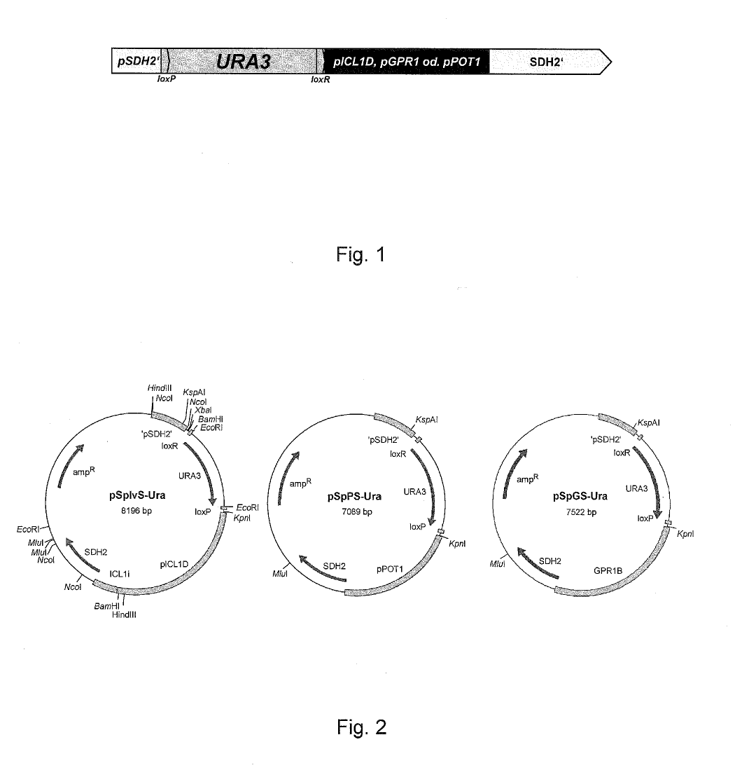

pUCBM21 (FIG. 2) served as a starting plasmid. In this, firstly the SDH2 promoter fragment pSDH2 (569 bp) amplified using the primers pSDH2-fw and pSDH2-ry was integrated into the NcoI cleavage site. By means of PCR, a fragment of the SDH2 ORF 1242 bp in size (primers: SDH2-fw and SDH2-rv) and a pICL1 fragment 776 bp in size (primers: pICL1-fw and pICL1-rv) were amplified and these were fused by means of overlap PCR (primers: overlap-fw and overlap-rv). After digestion using the restriction enzymes BamHI and EcoRI, this pICL1-SDH2 fragment was integrated into the vector contained in the pSDH2, likewise BamHI and EcoRI digested. In this construct, the pICL1 fragment 2.16 kb in size, which was obtained by means of KpnI and BamHI digestion from the plasmid p64PT (Gerber 1999), was cloned between the KpnI and BamHI cleavage sites. Finally, the integration of the loxR-URA3-loxP cassette into this plasmid with the ‘pSDH2-pICL1-SDH2’ cassette took place, which was digested with XbaI and KpnI.

The expression cassette 1.4 kb in size was isolated from the plasmid JMP113 (Fickers et al. 2003) by means of XbaI/KpnI digestion. Finally, the plasmid pSpIvS-Ura 8196 bp in size (FIG. 2) was obtained, which contained the expression cassette ‘pSDH2-loxR-URA3-loxP-pICL1-SDH2’ for the replacement of the native SDH2 promoter by the ICL1 promoter. This was isolated from the plasmid by KspAI/MluI digestion and used for the transformation by means of LiAc method in the uracil-auxotrophic recipient strain H222-SW2 (MATA ura3-302 ku70Δ-1572) (Werner 2008).

The plasmid pSpPS-Ura (FIG. 2) contained in ‘pSDH2-loxR-URA3-loxP-pPOT1-SDH2’ was constructed starting from pSpIvS-Ura. For this, pSpIvS-Ura was digested with the restriction enzymes KpnI and MluI and the fragment 4773 bp in size was isolated (using pSDH2 and URA3). Then the SDH2 fragment (838 bp, primers: SDH2-pPOT1-fw, SDH2-MluI-rv) obtained by means of PCR and the pPOT1 fragment (1538 bp, primers: KpnI-pPOT1-fw and pPOT1-SDH2-rv) were fused in an overlap PCR using the primers KpnI-pPOT1-fw and SDH2-MluI-ry to give a construct 2336 bp in size. This construct was digested with KpnI and MluI and ligated with the 4773 bp fragment of pSpIvS-Ura Finally, a pSpPS-Ura plasmid with a size of 7.1 kb was isolated, which contained the expression cassette ‘pSDH2-loxR-URA3-loxP-pPOT1-SDH2’ for the replacement of the native SDH2 promoter by the POT1 promoter. This expression cassette could also be isolated from the plasmid by means of KspAI/MluI digestion and be used for the transformation to the uracil-auxotrophic recipient strain H222-SW2 (MATA ura3-302 ku70Δ-1572) (Werner 2008).

Analogously to pSpPS-Ura, the plasmid pSpGS-Ura (FIG. 2) contained in PSDH2-loxR-URA3-loxP-GPR1B-SDH2 was constructed. GPR1B was isolated from the plasmid pTBS1 by means of PCR (primers: KpnI-pGPR1-fw and pGPR1-SDH2-rv, fragment size: 1971 bp). The SDH2 (838 bp, primers: SDH2-pGPR1-fw, SDH2-MluI-rv) obtained by means of PCR was fused in an overlap PCR with the primers KpnI-pGPR1-fw and SDH2-MluI-ry to give a construct 2769 bp in size. This construct was digested with KpnI and MluI and ligated with the 4773 bp fragment of pSpIvS-Ura Finally, a pSpGS-Ura plasmid with a size of 7.5 kb was isolated, which contained the expression cassette ‘pSDH2-loxR-URA3-loxP-GPR1B-SDH2’ for the replacement of the native SDH2-promoter by the GPR1B-promoter. The expression cassettes were sequenced for checking the sequence.

These expression cassettes were transformed into the uracil-auxotrophic recipient strain H222-SW2 (MATA ura3-302 ku70Δ-1572) (Werner 2008). Finally, the uracil-prototrophic transformants H222-AZ1 (pICL1-SDH2), and H222-AZ2 (pPOT1-SDH2) and H222-AZ3 (pGPR1-SDH2) were obtained. The integration of the corresponding expression cassettes was detected by means of PCR or Southern hybridization.

### 2. Combination of Overexpression of PYC1 and SDH2 Promoter Exchange or PYC1+ICL1 and SDH2 Promoter Exchange

The starting strain was H222-AZ2. Furthermore, starting from H222-AZ2, strains were constructed in which both PYC1 and the isocitrate lyase-coding gene ICL1 were present simultaneously overexpressed.

For the construction of a strain (H222-AZ8), in which both the SDH2 promoter replaced by the POT1 promoter and also the pyruvate carboxylase-coding gene PYC1 are present overexpressed, H222-AZ2 was used as the starting strain. To be able to employ this for further manipulations, a uracil auxotrophy had to be produced in H222-AZ2. The expression cassette for the replacement of the SDH2 promoter was constructed so that the marker gene URA3, which is essential for the endogenous cell uracil synthesis, was flanked by the so-called lox sites. To remove this marker gene again and thereby to achieve the loss of the uracil prototrophy, the so-called Crelox system was used. For this purpose, H222-AZ2 was transformed using the plasmid pUB4-Cre, which contained both a gene for the Cre recombinase and also a hygromycin B resistance-mediating gene. The cells were then selected on hygromycin B-containing complete medium and then selected on minimal medium with or without uracil. Transformants, in which with the aid of the Cre recombinase a recombination of the lox sites occurred, were no longer able on account of the loss of URA3 caused thereby to produce uracil and thus to grow on uracil-free medium.

One transformant was selected from these uracil-auxotrophic transformants and used for the further studies as H222-AZ2U. Subsequently, the integrative multicopy plasmid p64PYC1, which contained the gene PYC1 coding for pyruvate carboxylase in Y. lipolytica with its own promoter and terminator regions and also the URA3 allele ura3d4 as a multicopy marker gene and rDNA as an integration sequence, was transformed by means of lithium acetate method (Barth and Gaillardin 1996) into the uracil-auxotrophic strain H222-AZ2U. A selection of the uracil-prototrophic transformants took place on uracil-free minimal medium with glucose as the C source. From the transformants obtained, three were selected and checked with regard to the multiple integration of p64PYC1 by means of PCR and Southern Blot.

For the detection of the PYC activity, all three transformants and also the wild-type H222 were cultured in minimal medium containing glucose. After 4 hours, cells were harvested and mechanically disrupted. The determination of the PYC activity in the cell-free extract was carried out according to van Urk et al. (1989). Culturing was carried out analogously to the described culturing of the strains H222-AZ1 to H222-AZ3.

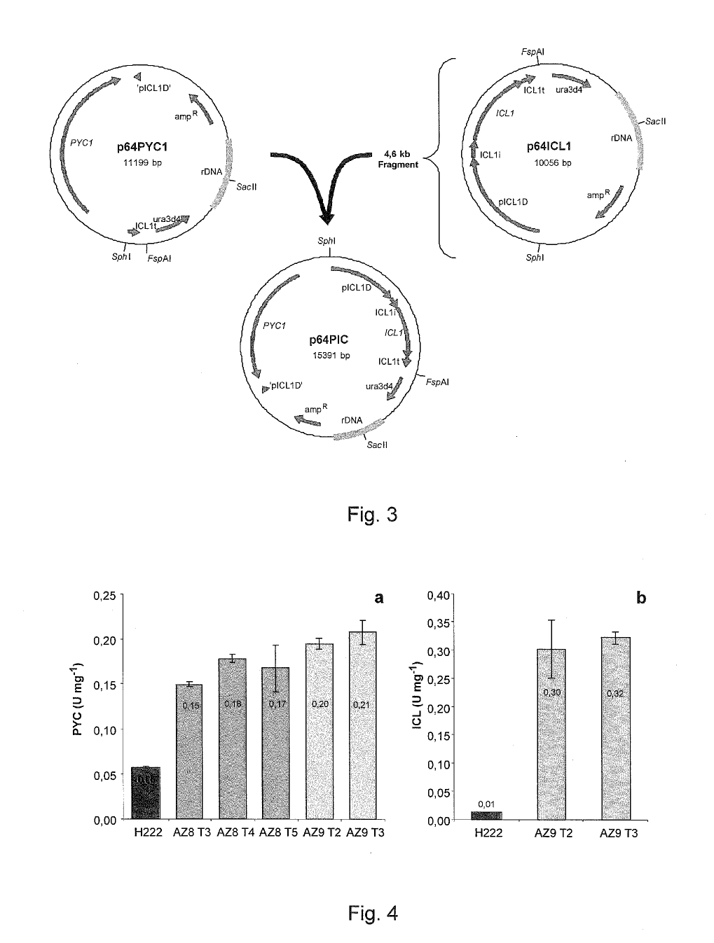

For the construction of a strain (H222-AZ9), in which both the SDH2 promoter replaced by the POT1 promoter and also PYC1 and ICL1 are present overexpressed, it was proceeded analogously to the construction of H222-AZ8 and a uracil-auxotrophic derivative of H222-AZ2 was used as a starting strain. The plasmid p64PIC to be transformed was constructed by restriction and ligation from the plasmids p64PYC1 and p64ICL1 (Kruse et al. 2004). For this purpose, the approx. 4.6 kb in size SphI-FspAI-fragment from p64ICL1 was isolated and integrated into the SphI-FspAI cleaved vector p64PYC1 (FIG. 3). The resulting plasmid p64PIC had a size of 15.4 kb and was linearized for the transformation in the SacII cleavage site. The integration was verified by means of PCR or Southern Blot. Two selected transformants were checked with regard to the PYC and ICL activity (Table 3) according to Dixon and Kornberg, (1959) and finally cultured in YNB medium.

TABLE 3
Composition of the reaction batch for the detemination of the ICL
activity. The principle of the assay is based on the measurement of the
increase in absorbance at 324 nm resulting from the formation of
glyoxylate phenylhydrazone (ε = 17 L mmol⁻¹ cm⁻¹) from glyoxylate
and phenylhydrazine.

| | Stock solution | | Final concentration | |
|---|---|---|---|---|
| Tris HCl, pH 7.0 | 100 | mM | 53.3 | mM |
| Cysteine HCl | 20 | mM | 2 | mM |
| MgCl₂ | 50 | mM | 5 | mM |
| D,L-Na isocitrate | 16.7 | mM | 1.67 | mM |
| Phenylhydrazine | | | | |
| HCl | 33 | mM | 3.3 | mM |

For characterization, three transformants of H222-AZ8 (T3-T5) as well as two transformants of H222-AZ9 (T2+T3) were selected. In all transformants the PYC activity was investigated and in the H222-AZ9 transformants additionally the activity of the ICL was investigated. The results of these activity measurements are shown in FIG. 4. All transformants of H222-AZ8 and also H222-AZ9 both showed an increased PYC activity (2.6-3.7-fold) in comparison to the wild-type H222. The transformants H222-AZ9 T2 and T3 moreover showed a 24-25-fold increase in the ICL activity compared with H222.

### 3. Deletion of JEN4

The inventors have carried out investigations on the characterization of the proteins in Y. lipolytica encoded by JEN orthologs. These investigations yielded information on the functioning of these JEN gene products as dicarboxylate importers using several specific substrates. Strains were constructed here in which individual JEN genes were deleted. On culturing these strains in some cases, particularly with the deletion of JEN4, relatively large amounts of extracellular succinate were detected.

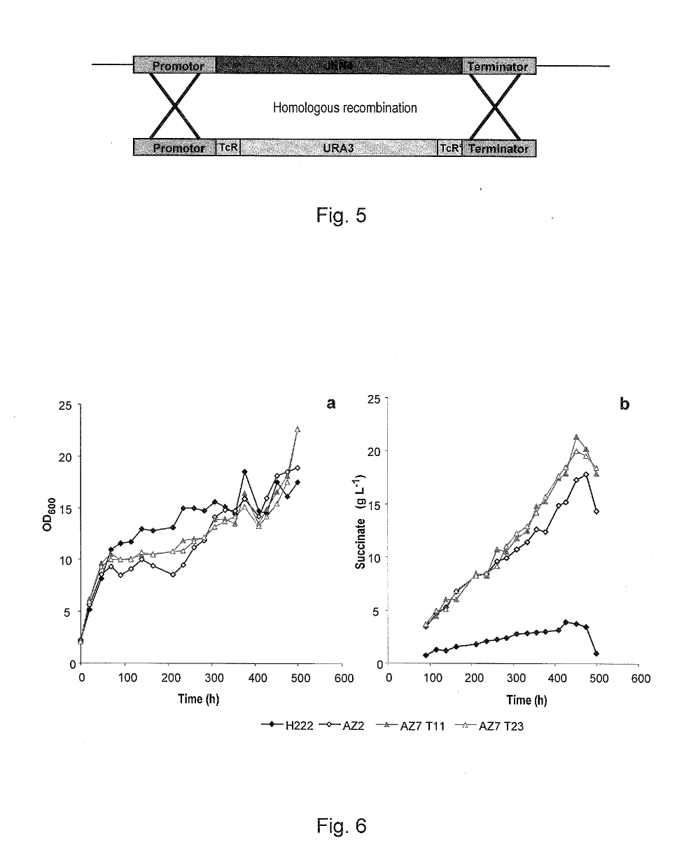

A JEN4 deletion cassette (approx. 4.5 kb) was transformed here in the uracil-auxotrophic Yarrowia lipolytica strain H222-AZ2U. The JEN4 deletion cassette (FIG. 5) contained the promoter and terminator regions of JEN4, between the URA3 flanked by TcR sequences (Hübner 2010). The deletion cassette was transformed by means of lithium acetate method. By homologous recombination in promoter and terminator region, a deletion of JEN4 should occur (FIG. 5). Two uracil-prototrophic transformants, in which the deletion of JEN4 was detected by means of PCR and Southern Blot, were isolated. Both transformants were cultured in YNB.

The two investigated transformants H222-AZ7 T11 and T23 behaved similarly to H222-AZ2 with regard to growth (FIG. 3). Both transformants moreover produced more succinate than the starting strains H222 and H222-AZ2 (FIG. 6, Tab. 2).

TABLE 4

| | Maximal succinate | | |
|---|---|---|---|
| | content | Q | q |
| Strains | [g L⁻¹] | [mg L⁻¹ h⁻¹] | [μg OD⁻¹ h⁻¹] |
| H222 | 3.6 ± 0.4 | 9.8 ± 0.6 | 0.60 ± 0.01 |
| AZ2 | 15.0 ± 4.0 | 31.8 ± 7.9 | 2.50 ± 0.68 |
| AZ7 T11 | 21.1 ± 0.4 | 50.7 ± 2.2 | 3.00 ± 0.09 |
| AZ7 T23 | 20.8 ± 1.1 | 54.7 ± 13.3 | 3.06 ± 0.07 |

Maximal succinate contents, volume-specific and biomass-specific product formation rate of the strains H222, H222-AZ7 T11 and T23 in YNB medium.
Culturing in YNB medium with 5% glycerol as C source.

### 4. Overexpression of PYC1

For the overexpression of pyruvate carboxylase (PYC) in Y. lipolytica, the integrative multicopy plasmid p64PYC was constructed by integration of the PCR-amplified ORF of the corresponding gene together with the individual promoter and terminator regions of approx. 1 kb into the host vector p64T.

Pyruvate carboxylase is encoded in Y. lipolytica by a gene (PVC1) (Flores and Gancedo 2005). As the region of the ORF of PYC1 (3844 bp) together with the associated in each case 1 kb in size promoter and 0.3 kb in size terminator region comprised a size of 5149 bp, this expression cassette was amplified in two steps in order to lower the introduction of possible sequence errors by the polymerase. The oligonucleotides PYCa_for and PYCa_rev amplified the region 2442 bp in size, which included the promoter region 1 kb in size and a part of the ORF of PYC1. The region 2822 bp in size, of the remaining ORF of PYC1 together with the terminator region 300 bp in size, was amplified by the oligonucleotides PYCb_for and PYCb_rev. For simplified integration into the expression vector p64T, a PaeI was added by the oligonucleotides PYCa_for and a BglII cleavage site by PYCb_rev, moreover the BglII cleavage site contained in the ORF was used for the vector integration and the fusion of the two fragments. The PYCa fragment 2442 bp in size (promoter region containing a part of the PYC-ORF) was digested using PaeI and BglII and cloned into the p64T vector likewise digested with PaeI and BglII. The resulting p64PYC1a vector was sequenced. The sequencing of the plasmid of the clone T22 revealed an error-free PYC1-ORF and an error-free promoter region was detected for the plasmid of the clone T97. An error-free p64PYC1a vector was achieved by the integration of the error-free PaeI-BamHI promoter region of p64PYC1a of the clone T97 into the p64PYC1a vector digested with PaeI and BamHI of the clone T22. In the resulting p64PYC1a vector, the BglII-PYCb fragment (part of the PYC1-ORF and the terminator region) was cloned and then sequenced. The vector p64PYC1 was obtained, which contained an error-free expression cassette (pPYC1-PYC1-PYC1t) for the overexpression of the gene coding pyruvate carboxylase. For the transformation in the Y. lipolytica strains H222-S4 and H222-AK7 (Δscs2), this vector was linearized with to SacII.

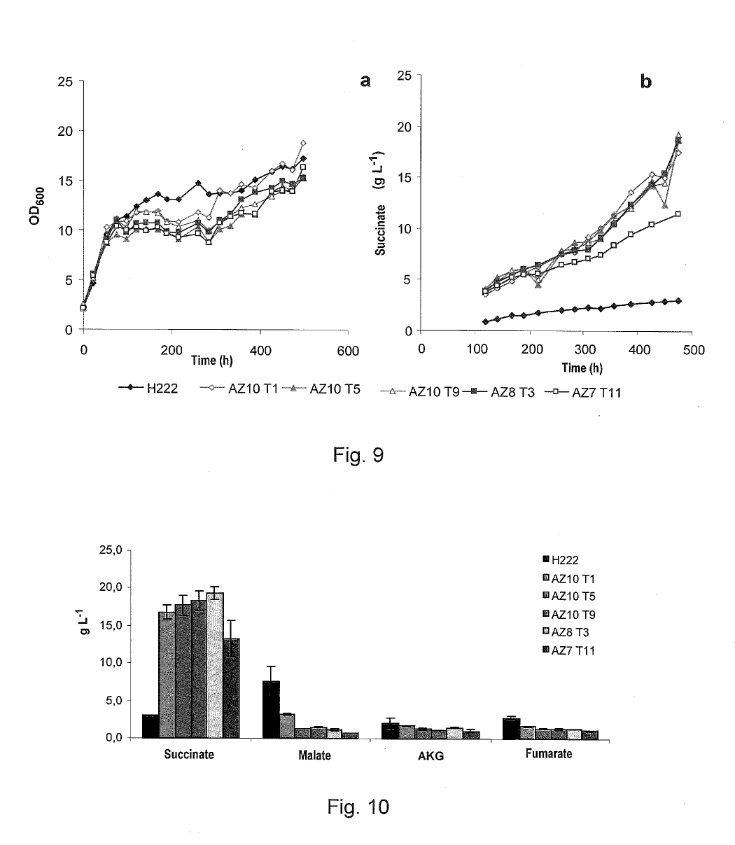

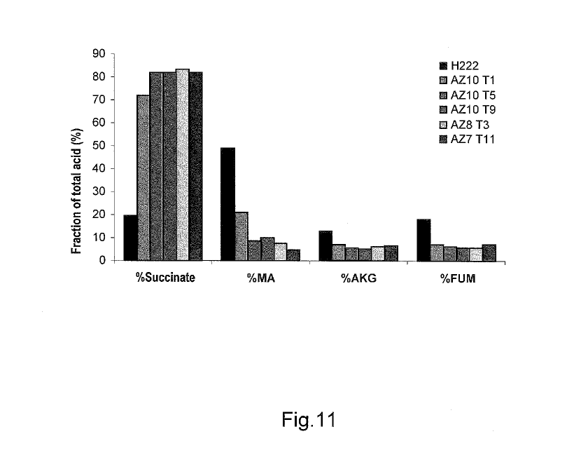

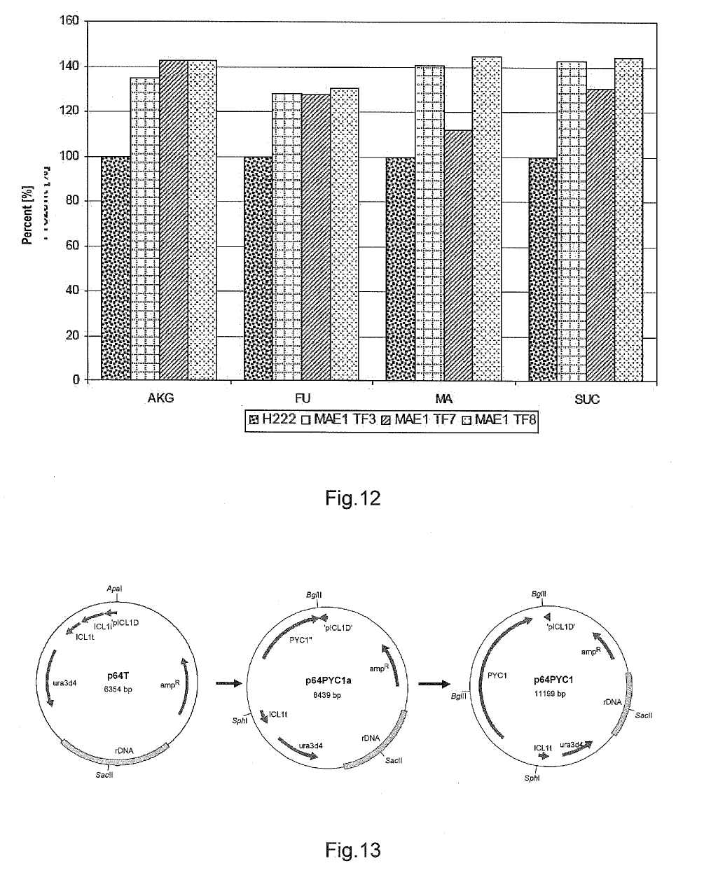

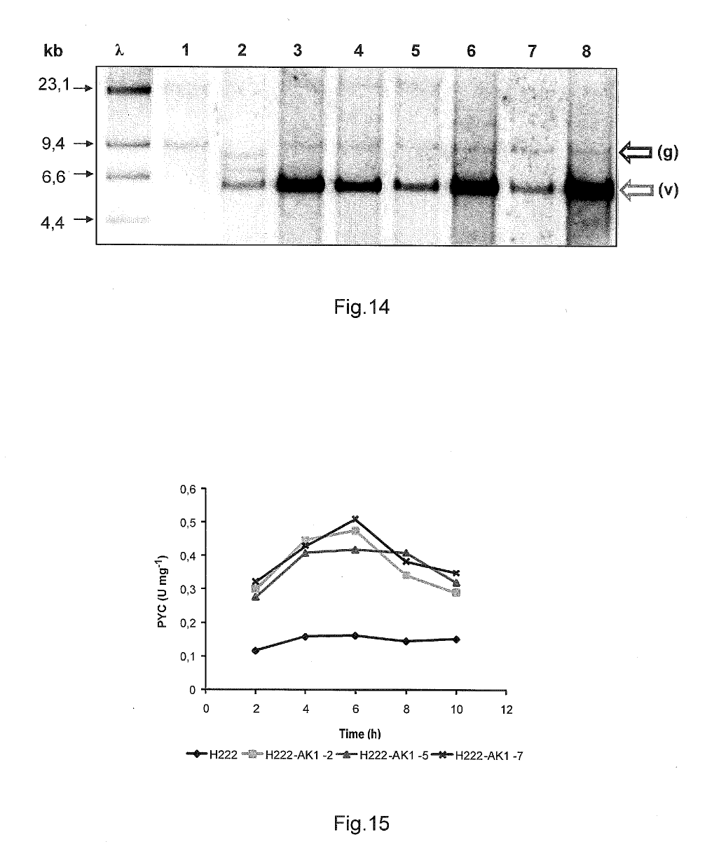

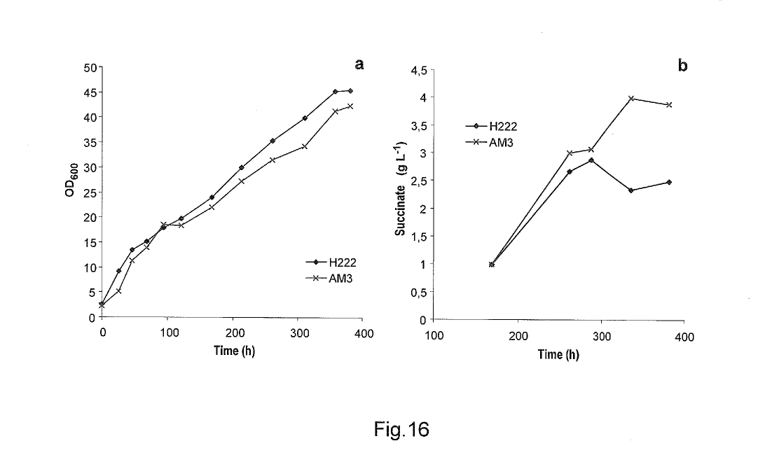

The integration of the expression cassette into the transformant H222-AK1 obtained was checked by means of Southern hybridization (FIG. 9). For the parent strain H222-S4, a band at 8.9 kb corresponding to the genomic PYC1-ORF was detected. The additionally detected band at 6.2 kb in the transformant served as proof for the integration of the multicopy vector p64PYC1. These bands showed an increased intensity compared with the genomic band of the comparison strain H222-S4, whereby a multiple integration of the corresponding expression cassette was confirmed. The transformants H222-AK1-2, H222-AK1-5 and H222-AK1-7 were selected for further investigations on the basis of their high vector band intensities.

The gene dose effect on the pyruvate carboxylase activity was investigated in the strains H222-AK1-2, H222-AK1-5 and H222-AK1-7.

For the determination of the PYC activity, all selected transformants were cultured in 100 ml of minimal medium containing 1% glucose. After harvesting a sample of the culture, the yeast cells were disrupted by means of glass beads. The determination of the PYC activity was carried out in the cell-free extract according to the method of van Urk et al. (1989). The composition of the reaction batch is listed in the following table. The photometric measurement was carried out at 340 nm.

| | Stock solution | | Final concentration | |
|---|---|---|---|---|
| Tris HCl, | 1 | M | 100 | mM |
| pH 7.8 | | | | |
| MgSO₄ | 100 | mM | 6.7 | mM |
| KHCO₃ | 400 | mM | 40 | mM |
| Pyrazole | 15 | mM | 1.5 | mM |
| NADH | 1.5 | mM | 0.15 | mM |
| Acetyl-CoA | 10 | mM | 90 | μM |
| Pyruvate | 100 | mM | 10 | mM |
| Malate | 6 | U μl⁻¹ | 6 | U |
| DH | | | | |
| ATP | 33 | mM | 3.3 | mM |

The increased gene dose of the gene coding PYC showed a positive effect on the specific PYC activities of the strains investigated. The specific activities for the transformants H222-AK1-2 and H222-AK1-7 reached a maximum after 6 h. In contrast to that, no discernible maximum of the specific enzyme activity was detected for the transformant H222-AK1-5 and for the wild-type H222. The transformants H222-AK1-2 and H222-AK1-7 showed the highest specific PYC activities in comparison to the strains further investigated at 0.48±0.04 U/mg and at 0.51±0.02 U/mg, the enzyme activities of which were determined at 0.42±0.1 U/mg (H222-AK1-5) and at 0.16±0.06 U/mg (H222). The strain H222-AK1-7, in addition to the highest specific activity, showed a growth comparable with the wild-type and was thus selected for the further investigations. This transformant H222-AK1-7 is designated below as transformant H222-AK1 (=H222-AM3).

In comparison to the wild-type strain H222, the strain with increased PYC activity showed moderate differences with respect to growth and production behavior. The wild-type H222 maximally produced 3.3±0.02 g L⁻¹ of succinate with an average productivity of 5.4±0.6 mg L⁻¹ h⁻¹. For H222-AM3, it was possible to detect slightly increased succinate amounts of 4.1±0.1 g L⁻¹.

5. Combination of JEN4 Deletion with Overexpression of PYC1

Since both overexpression of PYC1 as well as the deletion of JEN4 starting from H222-AZ2 (with SDH2 promoter exchange) led to an increased succinate production, strains for succinate production were constructed in which these three modifications are contained combined.

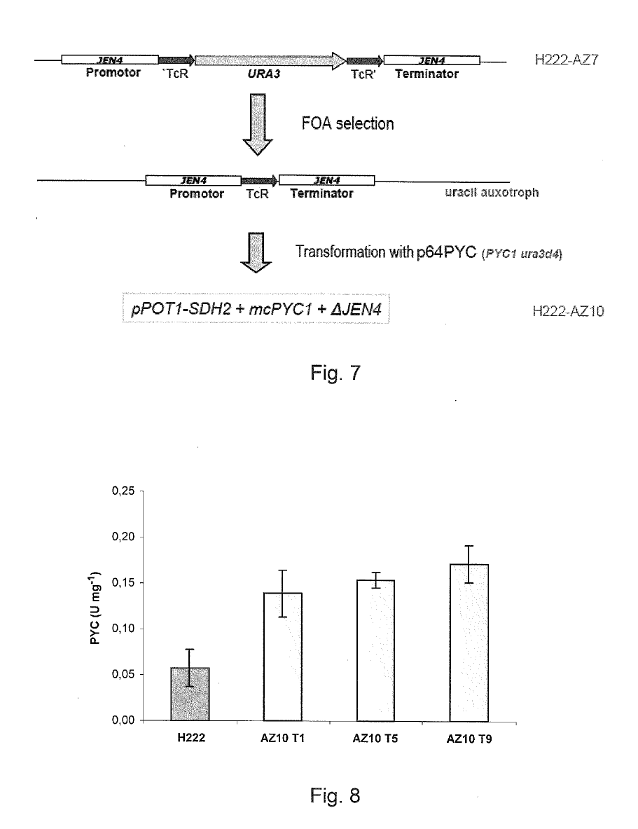

The construction of this new strain (H222-AZ10) took place starting from H222-AZ7 (4JEN4). For this purpose, a uracil auxotrophy was introduced in H222-AZ7 by means of FOA selection. The strategy is shown in FIG. 7. FOA (5′-fluoroorotic acid) is converted to 5-fluorouracil by oritidine 5′-phosphate decarboxylase, which is encoded by URA3. 5-Fluorouracil is cytotoxic for the cell. Therefore, in the presence of FOA, only cells can grow in which no URA3 is present or in which URA3 was removed by recombination of the flanking TcR sequences. For this purpose, 10³ cells of H222-AZ7 were streaked out on FOA- and uracil-containing minimal medium with glucose and the colonies obtained were checked for uracil auxotrophy by means of replica test. The loss of the URA3 was moreover detected by means of PCR. A uracil auxotroph (H222-AZ7U) was selected from the colonies tested and transformed with the integrative PYC1 multicopy vector p64PYC1. After one to two weeks transformants were isolated, which were checked by means of PCR for the integration of the vector. All transformants tested were positive. From these positive transformants, 3 were selected and tested with regard to the PYC activity. For this purpose, these 3 transformants as well as the wild-type were cultured in minimal medium with glucose. After 4 hours, a sample was taken for the determination of the PYC activity and the activity of the pyruvate carboxylase was determined.

Three selected transformants were investigated with regard to the PYC activity and succinate production. The results of the activity measurements are shown in FIG. 8. All transformants show a two- to three-times increased PYC activity in comparison to the wild-type.

The three tested transformants were cultured in YNB medium in order to investigate the effects on the succinate production.

Growth behavior and succinate production of H222 (wild-type) and the new transformants H222-AZ10 T1, T5 and T9 are shown in FIG. 9. As a comparison, they were moreover accompanied by the strains H222-AZ7 T11 and H222-AZ8 T3. The average values of all experiments carried out are summarized in Table 5.

TABLE 5
Average maximal succinate contents, volume-specific productivities
and biomass-specific product formation rates of the strains H222
(wild-type), H222-AZ10 T1, T5, T9, H222-AZ8 T3 and H222-AZ7 T11.
Culturing in shaker flasks at 28° C., 220 rpm in 150 ml of YNB medium
with 5% glycerol as C source (+3% after 168 h) in two experimental runs.

| | Succinate | Q |
|---|---|---|
| Strains | [g L⁻¹] | [mg L⁻¹ h⁻¹] |
| H222 | 3.0 ± 0.0 | 6.0 ± 0.1 |
| AZ10 T1 | 16.8 ± 1.0 | 34.8 ± 3.1 |
| AZ10 T5 | 17.7 ± 1.3 | 36.1 ± 3.4 |
| AZ10 T9 | 18.3 ± 1.3 | 36.8 ± 4.4 |
| AZ8 T3 | 19.3 ± 0.9 | 45.5 ± 2.7 |
| AZ7 T11 | 13.2 ± 2.4 | 36.6 ± 10.5 |

Additionally to the succinate production, the by-product spectrum of the organic acids malate, AKG and fumarate was also considered. All modified strains showed markedly reduced contents of malate, AKG and fumarate, however, in comparison to the wild-type (Tab. 6).

TABLE 6
Maximally formed product amounts of the organic acids malate (MA),
α-ketoglutarate (AKG) and fumarate (FA) on culturing the strains
Y. lipolytica H222, H222-AZ10 T1, T5 and T9 in YNB medium.
Culturing in YNB medium with 5% glycerol as C source.

| | MAₘₐₓ | AKGₘₐₓ | FAₘₐₓ |
|---|---|---|---|
| | [g L⁻¹] | [g L⁻¹] | [g L⁻¹] |
| H222 | 7.5 ± 2.1 | 2.0 ± 0.7 | 2.8 ± 0.2 |
| AZ10 T1 | 3.2 ± 0.1 | 1.7 ± 0.1 | 1.7 ± 0.1 |
| AZ10 T5 | 1.3 ± 0.0 | 1.3 ± 0.2 | 1.3 ± 0.1 |
| AZ10 T9 | 1.5 ± 0.0 | 1.2 ± 0.0 | 1.3 ± 0.1 |

### 6. Deletion of MAE1

In a similar manner as described above, the gene of the carboxylate transporter Mae1 was deleted in Yarrowia lipolytica. The deletion cassettes and plasmids used are found in the table shown at the beginning. The deletion of the SpMAE1-homologous gene MAE1, coding for a putative dicarboxylate transporter, took place in the deletion strain H222-SW2ΔMAE1. During the culturing of the H222-SW2ΔMAE1 transformants 3, 7 and 8, the accumulated organic acids α-ketoglutarate, fumarate, malate, pyruvate and succinate were investigated in comparison to the starting strain H222. Additionally, the determination of dry biomasses formed furthermore took place during the culturing.

The dry biomass of the ΔMAE1-transformants formed on culturing in 10% glycerol was lower by 40% in comparison to the reference strain H222, but showed hardly any differences among themselves in comparison to the transformants. This marked difference in growth can result from a reduced uptake of the tricarboxylic acid cycle intermediates α-ketoglutarate, fumarate, malate and succinate, which are starting materials of many biosyntheses, such as, for example, for amino or fatty acids. On consideration of the acid production, all transformants showed an increase in the production of the organic acids α-ketoglutarate, fumarate, malate and succinate investigated. On average, maximally 30% more AKG, fumarate and succinate, and also 40% more malate (TF7 only 10%), were formed here. The results are found in Table 7. The average values of the maximal amounts of malate, succinate, α-ketoglutarate and fumarate of the transformants 3, 7, 8 of H222-SW2ΔMAE1 are shown compared with reference strain H222 on culturing in production medium according to Tabuchi et al. (1981). SA ΔMAE1=standard deviation of the maximal values of the 3 transformants:

| | Organic acids in g L⁻¹ | | | |
|---|---|---|---|---|
| Strain | α-Ketoglutarate | Fumarate | Malate | Succinate |
| H222 | 8.7 | 3.6 | 4.8 | 2.9 |
| ΔMAE1 | 12.2 | 4.7 | 6.4 | 4.0 |
| SA ΔMAE1 | 0.31 | 0.05 | 0.70 | 0.17 |

### LITERATURE

Arikawa et al. (1999) Isolation of sake yeast strains possessing various levels of succinate- and/or malate-producing abilities by gene disruption or mutation. Journal of Bioscience and Bioengineering Volume 87, Issue 3, 1999, Pages 333-339
Ghezzi D, Goffrini P, Uziel G, Horvath R, Klopstock T, Lochmüller H, D'Adamo P, Gasparini P, Strom T M, Prokisch H, Invernizzi F, Ferrero I, Zeviani M. (2009) SDHAF1, coding a LYR complex-II specific assembly factor, is mutated in SDH-defective infantile leukoencephalopathy. Nat Genet. 2009 June; 41(6):654-6. Epub 2009 May 24.
Kubo Y, Takagi H and Nakamori S, (2000). Effect of gene disruption of succinate dehydrogenase on succinate production in a sake yeast strain. Journal of Bioscience and Bioengineering Volume 90, Issue 6, 2000, Pages 619-624
Raab A and Lang (2011), Oxidative versus reductive succinic acid production in the yeast saccharomyces cerevisiae, Bioengineered Bugs 2(2) 2011, 120-123
Mauersberger, S., Wang, H. J., Gaillardin, C., Barth, G. and Nicaud, J. M. (2001). Insertional mutagenesis in the n-alkane-assimilating yeast Yarrowia lipolytica: generation of tagged mutations in genes involved in hydrophobic substrate utilization. J Bacteriol 183(17): 5102-5109.
Fickers, P., Le Dall, M. T., Gaillardin, C., Thonart, P. and Nicaud, J. M. (2003). New disruption cassettes for rapid gene disruption and marker rescue in the yeast Yarrowia lipolytica. J Microbiol Methods 55(3): 727-737.
Werner, S. (2008). Herstellung and Charakterisierung eines Stammes der Hefe Yarrowia lipolytica with erniedrigter heterologer Rekombination [Preparation and characterization of a strain of the yeast Yarrowia lipolytica with decreased heterologous recombination]. Thesis. TU Dresden.
Barth, G. and Gaillardin, C. (1996). Yarrowia lipolytica. Nonconventional Yeasts in Biotechnology. K. Wolf. Berlin, Heidelberg, New York, Springer-Verlag van Urk, H., Schipper, D., Breedveld, G. J., Mak, P. R., Scheffers, W. A. and van Dijken, J. P. (1989). Localization and kinetics of pyruvate-metabolizing enzymes in relation to aerobic alcoholic fermentation in Saccharomyces cerevisiae CBS 8066 and Candida utilis CBS 621. Biochim Biophys Acta 992(1): 78-86.
Gerber, J. (1999) Untersuchungen zur Optimierung des Elktronentransportsystems für die Cytochrom P450 katalysierte Biotransformation von Steroiden in Yarrowia lipolytica [Investigations on the optimization of the electron transport system for cytochrome P450-catalyzed biotransformation of steroids in Yarrowia lipolytica]. Thesis. TU Dresden
Dixon, H. G. and Kornberg, H. L. (1959). Assay method for key enzymes of the glyoxylate cycle. Biochem J 72: 3P
Kruse, K., Förster, A., Juretzek, T., Mauersberger, S. and Barth, G. (2004). Verfahren zur biotechnologischen Herstellung von Citronensäure mit einer genetisch veränderten Hefe Yarrowia lipolytica [Process for the biotechnological production of citric acid using a genetically modified yeast Yarrowia lipolytica]. Patent: DE 10333144A1
Tabuchi, T., Sugisawa, T., Ishidori, T., Nakahara, T. and Sugiyama, J. (1981). Itaconic acid fermentation by a yeast belonging to the genus Candida. Agric Biol Chem 45: 745-479.

1-11. (canceled)

12. An isolated fungal strain, having at least one genetic modification which leads to a decrease in the activity of at least one carboxylic acid transporter.

13. The fungal strain according to claim 12, wherein the fungal strain has increased extracellular carboxylic acid production compared to a fungal strain lacking the at least one genetic modification.

14. The fungal strain according to claim 12, wherein the fungal strain is a member of the phylum Ascomycota.

15. The fungal strain according to claim 12, wherein the fungal strain is selected from the group consisting of Saccharomyces, Schizosaccharomyces, Wickerhamia, Debayomyces, Hansenula, Hanseniospora, Pichia, Kloeckera, Candida, Zygosaccharomyces, Ogataea, Kuraishia, Komagataella, Yarrowia, Metschnikowia, Williopsis, Nakazawaea, Kluyveromyces, Cryptococcus, Torulaspora, Torulopsis, Bullera, Rhodotorula, Sporobolomyces, Pseudozyma, Saccharomycopsis, Saccharomycodes, Aspergillus, Penicillium, Rhizopus, Trichosporon, and Trichoderma.

16. The fungal strain according to claim 12, wherein the fungal strain belongs to the species Yarrowia lipolytica.

17. The fungal strain according to claim 12, wherein the carboxylic acid transporter is selected from the group consisting of JEN transporters and MAE transporters.

18. The fungal strain according to claim 12, wherein the fungal strain has at least one further genetic modification selected from the group consisting of reduction of the activity or expression of succinate dehydrogenase (SDH), increase in the activity or expression of pyruvate carboxylase (PYC), and increase in the activity or expression of isocitrate lyase (ICL).

19. A method of increasing extracellular carboxylic acid production in a fungal strain comprising genetically modifying the fungal strain by:
a) inhibiting or reducing the expression of a gene coding for a carboxylic acid transporter;
b) expressing a dysfunctional or decreased activity carboxylic acid transporter; and/or
c) inhibiting or reducing the activity of an expressed carboxylic acid transporter,
thereby producing a fungus having increased extracellular carboxylic acid production compared to a fungal strain that is not genetically modified by at least one of the inhibiting or expressing steps.

20. The method according to claim 19, wherein the fungal strain is a member of the phylum Ascomycota.

21. The method according to claim 19, wherein the fungal strain is selected from the group consisting of Saccharomyces, Schizosaccharomyces, Wickerhamia, Debayomyces, Hansenula, Hanseniospora, Pichia, Kloeckera, Candida, Zygosaccharomyces, Ogataea, Kuraishia, Komagataella, Yarrowia, Metschnikowia, Williopsis, Nakazawaea, Kluyveromyces, Cryptococcus, Torulaspora, Torulopsis, Bullera, Rhodotorula, Sporobolomyces, Pseudozyma, Saccharomycopsis, Saccharomycodes, Aspergillus, Penicillium, Rhizopus, Trichosporon, and Trichoderma.

22. The method according to claim 19, wherein the fungal strain belongs to the species Yarrowia lipolytica.

23. The method according to claim 19, wherein the carboxylic acid transporter is selected from the group consisting of JEN transporters and MAE transporters.

24. The method according to claim 19, wherein the fungal strain is further modified, the further genetic modification selected from the group consisting of reducing the activity or expression of succinate dehydrogenase (SDH), increasing the activity or expression of pyruvate carboxylase (PYC), and increasing the activity or expression of isocitrate lyase (ICL).

25. The method according to claim 24, wherein reducing the activity or expression of SDH is caused by replacing the native promoter of SDH2 with an inducible promoter or by using a deletion cassette.

26. The method according to claim 24, wherein increasing the activity or expression of PYC is caused by overexpressing PYC1.

27. The method according to claim 24, wherein increasing the activity or expression of ICL is caused by overexpressing ICL1.

28. A method of producing carboxylic acid, comprising culturing an isolated fungal strain, the fungal strain having at least one genetic modification which leads to a decrease in the activity of at least one carboxylic acid transporter.

29. The method according to claim 28, wherein the fungal strain is selected from the group consisting of Saccharomyces, Schizosaccharomyces, Wickerhamia, Debayomyces, Hansenula, Hanseniospora, Pichia, Kloeckera, Candida, Zygosaccharomyces, Ogataea, Kuraishia, Komagataella, Yarrowia, Metschnikowia, Williopsis, Nakazawaea, Kluyveromyces, Cryptococcus, Torulaspora, Torulopsis, Bullera, Rhodotorula, Sporobolomyces, Pseudozyma, Saccharomycopsis, Saccharomycodes, Aspergillus, Penicillium, Rhizopus, Trichosporon, and Trichoderma.

30. The method according to claim 28, wherein the fungal strain belongs to the species Yarrowia lipolytica.

31. The method according to claim 28, wherein the fungal strain has at least one further genetic modification selected from the group consisting of reduction of the activity or expression of succinate dehydrogenase (SDH), increase in the activity or expression of pyruvate carboxylase (PYC), and increase in the activity or expression of isocitrate lyase (ICL).


SEQUENCE LISTING (14 sequences)

SEQ ID NO: 1 - DNA, 4384 bp, Artificial Sequence
  >JEN2_DK pUC_JEN2_PUT_rev cut MunI..2293 to Van91I..6677
aattgcgtgctggggcgttatttatgcactagtacacgggtatttaaatacaggttggatgtgattcgagtgggcgtaggtacctatatatgctcaacttctagacagtatcatcatgttaacgaggtgtaataggctcggagattcatcgggcccttatcaatatctatcttaataaaagtgtctcatctcggcctgcctgctgccatcagtaaatgactcaagcggagaattctcgggaattctatttgagatggacaatgatcccatcacacggtgtgcagccctggaggcggcaaaaagtaccaatcctggagagctatgcaggctcccccaacactactgattgaacagtgaagaataagcaacaccaaataagttaagctaataaatagggtagagatagggtgcgaggctggagcagctgtggtgtgttagataaggggaaatagtgctaggcgataagcctgttatcagtggctttcaaacttacggtacttgtagtcgcgttctgacgtctttcccatactaataacgcacttaccgtcatcaagtgggtttttgtacacatgccacaatcccccgtcccgtcaccccgtcccgtcacccgaaccaactctgcaacctgtcggctctcataccaccaaacaggaacaagaaacaacttgaaatacggttcatgcattttccccacaccgacaagcatctgaataaacctaatcagtcttgaagctggcaagatagtctctggaggagatcggatctactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctcctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccagaattctactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctggatctccagttctgtacagccgatcccggggacccttgtagaactctctccaggcctcacccttgttgaccttctcgtcctcctggatcccaaggctcgattctcatacgacgcatatgtatgtactatttgacggccaccaaaagtagctaggcacgtactgtatcataccagaccttatccgagagtcgaccctcttgttctaatcatacatctcgaattatgagctatggagaaaaatctgctacttgtagtaatctagcatcttgccgttccccaaatacgacgttctcggggttaatattatccccgacaccgttcttagactatggctccttacgctccacatatagtagcatcgacaaacgagcgcaagtataaagtacgaaacaagggtatcagtatgcttcatgttatagcgaggaagatgactaagccgtaattgagaaagctcactcttgcgagattcagagcacactaataatagtattaatgattaaaccaactgtcagattttgagcatgtggtaaacgtcgttttgtaattctgttgttctgtgagataattacagtaggtcttatcatatcaacaacaccccatagataagagtacgtacagtacgacatacgagtataccttggcagttgtcccgtctccaccgtaagtagctatttcaacagggacttcgaaagtcaacttttgtacaatcgtaattattgcttgtatacagcctcatctatccaatagtcatgtgctgaagagccaccatctccgttagcgcgttacatagtatactggcagcctaccttatcctaccaatagtctcgtggtgaaccagtta

SEQ ID NO: 2 - DNA, 6962 bp, Artificial Sequence
  pUC_JEN2_PUT_rev Ligation of inverted pUCLys2_DK2* into pUC_JEN1_P_T*
gcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcccgtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtactgggtccacttgtgaatgcggggttgttgagcaaccctcttgccttttcttggccaccccatactgcttctgaactggtctgatggggatctgacgcggtgtctgagtttggtgtcttactgcttgtaggagaatcctcagacgagtacatgcggtagggtttctacacgctcaattgcgtgctggggcgttatttatgcactagtacacgggtatttaaatacaggttggatgtgattcgagtgggcgtaggtacctatatatgctcaacttctagacagtatcatcatgttaacgaggtgtaataggctcggagattcatcgggcccttatcaatatctatcttaataaaagtgtctcatctcggcctgcctgctgccatcagtaaatgactcaagcggagaattctcgggaattctatttgagatggacaatgatcccatcacacggtgtgcagccctggaggcggcaaaaagtaccaatcctggagagctatgcaggctcccccaacactactgattgaacagtgaagaataagcaacaccaaataagttaagctaataaatagggtagagatagggtgcgaggctggagcagctgtggtgtgttagataaggggaaatagtgctaggcgataagcctgttatcagtggctttcaaacttacggtacttgtagtcgcgttctgacgtctttcccatactaataacgcacttaccgtcatcaagtgggtttttgtacacatgccacaatcccccgtcccgtcaccccgtcccgtcacccgaaccaactctgcaacctgtcggctctcataccaccaaacaggaacaagaaacaacttgaaatacggttcatgcattttccccacaccgacaagcatctgaataaacctaatcagtcttgaagctggcaagatagtctctggaggagatcggatctactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctcctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccagaattctactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctggatctccagttctgtacagccgatcccggggacccttgtagaactctctccaggcctcacccttgttgaccttctcgtcctcctggatcccaaggctcgattctcatacgacgcatatgtatgtactatttgacggccaccaaaagtagctaggcacgtactgtatcataccagaccttatccgagagtcgaccctcttgttctaatcatacatctcgaattatgagctatggagaaaaatctgctacttgtagtaatctagcatcttgccgttccccaaatacgacgttctcggggttaatattatccccgacaccgttcttagactatggctccttacgctccacatatagtagcatcgacaaacgagcgcaagtataaagtacgaaacaagggtatcagtatgcttcatgttatagcgaggaagatgactaagccgtaattgagaaagctcactcttgcgagattcagagcacactaataatagtattaatgattaaaccaactgtcagattttgagcatgtggtaaacgtcgttttgtaattctgttgttctgtgagataattacagtaggtcttatcatatcaacaacaccccatagataagagtacgtacagtacgacatacgagtataccttggcagttgtcccgtctccaccgtaagtagctatttcaacagggacttcgaaagtcaacttttgtacaatcgtaattattgcttgtatacagcctcatctatccaatagtcatgtgctgaagagccaccatctccgttagcgcgttacatagtatactggcagcctaccttatcctaccaatagtctcgtggtgaaccagttaatggaatccgggtgccaaggagctactagtacttaggtttgggcttcgaggtttttgtaccccgacgatctcgacgcggactcagtgacagcttcaagtgcgtcactgagtcaggcaaaacaatagaagagaaggactgtcaacggctaactattgttataatttaagcaactgctaaaatcaaggctaaaatgggccttaattcaaagctgtgtaacattgctgatacacttttacacgcgccagggatgaggggagataagacatgatctgcctgtagacggc

SEQ ID NO: 3 - DNA, 4759 bp, Artificial Sequence
  >JEN1_DK pUC_J1_PUT for 304 to 5062
gggttattccttccgagatgtgccggccaagaaggggttttgttatggcgtagacatgattccaatacagtacgagtacagtacccaaaaacgcaagatcctcttgaagagctccactcctcctctccttagaccgtaattgtcaccctgaaacctctccgcttatttgtctgtcttcccgttgctttcccagcctcgcatgcctatttcctactctaacttacagtacatagtatggacgaggaaaagaagtatacgacacaagtcaagaggcgcgtgcaggaccaagtaaacccgcttttgcactagtaatactaattcctttgtctggcataagtggggaagcgctaggcaactcaacgagcccatgacgggcaagagcgagcaaatcatagcgcggcatctacaaaattagcggaacttaatttgaaaggtgaaggagggctgtcaggagccaagttcctagccaagccagacacgatacatacgctggaagagctcgaaaagggaataatactgactcccccctttattacaacttcccttgtgggtcaggtgggctctaacaacaacttggacaacccgtgtgtttctcttggaaaatatggcatgcacggctaaaccctgtacaccctggcgcgttaatcacgccaagaagacaagcagtatgggaccggaaattccggggaaaatgcgtcttcatgtgtcatattctcttagttagaccgtgccaagaacccaaatgccaagaatccaagccttggtgacaagagcccaagacgaatacgatgtaccaagataccaagcgatgagcttgggggacgggcgccagaaagagcatcgtgaaagaaaaaaaaatgttcaacttatgttttaaggagaagagtcactctgaaaacccgcccgctcttgtgaactctgaccttaggacccggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatccacactcgtacaagggtactgaatgtaaggtacaatgtaccagtaaagtgcaagtacggtggatttggtcaacgaatgtccaagcatgccttcaagtgggaaacactacattactgtactatcatctgaagtctacaagtacagtacagtatgtacatacagtactgtacttatgccattgggctcgttgcactacgacggacacggaaacagagcagcttaaaagcaagtacttcttacataacattcggaggttccccgttgcaaatgtgagcttcatagtacaagtaagatgttcctaaaatgtattacactggacaccagttgcgaatttcaatacccagcccaaaaattgaattaagcttcaatgcccgtctgtgcagaattacgaactacagttcgggcaaacaaagaaaacgtcgtgatatgaaccctattgtgagacagtatctactgagactggaggagtctaggtgctccctctgacgctactggttcagaactgactgccggtgcgaggtgaaatttgttgtcatttaaccccattctttatcattgtattttatcaatacgaatccagtaacagaaggtaataaaccccactcatggccattgctcacaaactcctacaaagtatattatcgtaccaccatttgttatacatacacatacattataggctattgaaagaattaatctcatacgatacaaaagcaactgttggtatcctttcgcagtttgttcaactcactcaattcactgataatcgtcaactaatggactacacacaaccctacacctgctacaagctcgacggcacagatgatctagtacagtcaccttttgttgaagtcgcccttaaaccaaagaagggtatcaaaatcagagctctgatatcaccccattcaaatgtttctctggtagacagaaggactctcatcgaacacaggctc

SEQ ID NO: 4 - DNA, 7323 bp, Artificial Sequence
  >pUC_JEN1_PUT for Ligation of pUCLys2_DK2* into pUC_Jen1_PT*
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttccatgggatatcgcatgcctgcagagctctagagtcgacgggcccggtaccgcggccgtatcaccgggttattccttccgagatgtgccggccaagaaggggttttgttatggcgtagacatgattccaatacagtacgagtacagtacccaaaaacgcaagatcctcttgaagagctccactcctcctctccttagaccgtaattgtcaccctgaaacctctccgcttatttgtctgtcttcccgttgctttcccagcctcgcatgcctatttcctactctaacttacagtacatagtatggacgaggaaaagaagtatacgacacaagtcaagaggcgcgtgcaggaccaagtaaacccgcttttgcactagtaatactaattcctttgtctggcataagtggggaagcgctaggcaactcaacgagcccatgacgggcaagagcgagcaaatcatagcgcggcatctacaaaattagcggaacttaatttgaaaggtgaaggagggctgtcaggagccaagttcctagccaagccagacacgatacatacgctggaagagctcgaaaagggaataatactgactcccccctttattacaacttcccttgtgggtcaggtgggctctaacaacaacttggacaacccgtgtgtttctcttggaaaatatggcatgcacggctaaaccctgtacaccctggcgcgttaatcacgccaagaagacaagcagtatgggaccggaaattccggggaaaatgcgtcttcatgtgtcatattctcttagttagaccgtgccaagaacccaaatgccaagaatccaagccttggtgacaagagcccaagacgaatacgatgtaccaagataccaagcgatgagcttgggggacgggcgccagaaagagcatcgtgaaagaaaaaaaaatgttcaacttatgttttaaggagaagagtcactctgaaaacccgcccgctcttgtgaactctgaccttaggacccggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatccacactcgtacaagggtactgaatgtaaggtacaatgtaccagtaaagtgcaagtacggtggatttggtcaacgaatgtccaagcatgccttcaagtgggaaacactacattactgtactatcatctgaagtctacaagtacagtacagtatgtacatacagtactgtacttatgccattgggctcgttgcactacgacggacacggaaacagagcagcttaaaagcaagtacttcttacataacattcggaggttccccgttgcaaatgtgagcttcatagtacaagtaagatgttcctaaaatgtattacactggacaccagttgcgaatttcaatacccagcccaaaaattgaattaagcttcaatgcccgtctgtgcagaattacgaactacagttcgggcaaacaaagaaaacgtcgtgatatgaaccctattgtgagacagtatctactgagactggaggagtctaggtgctccctctgacgctactggttcagaactgactgccggtgcgaggtgaaatttgttgtcatttaaccccattctttatcattgtattttatcaatacgaatccagtaacagaaggtaataaaccccactcatggccattgctcacaaactcctacaaagtatattatcgtaccaccatttgttatacatacacatacattataggctattgaaagaattaatctcatacgatacaaaagcaactgttggtatcctttcgcagtttgttcaactcactcaattcactgataatcgtcaactaatggactacacacaaccctacacctgctacaagctcgacggcacagatgatctagtacagtcaccttttgttgaagtcgcccttaaaccaaagaagggtatcaaaatcagagctctgatatcaccccattcaaatgtttctctggtagacagaaggactctcatcgaacacaggctctttcagcacctcgaaatcgggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcccgtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 5 - DNA, 4393 bp, Artificial Sequence
  >JEN6_DK pUC_JEN6_PUT_for cut MunI..3038 to BsmI..642
aattggggatacatcaaggacgtctttgatcacaagcgacatggtaaggaggagtatgacttttcagaggtggagaagcacggtagttgatgaaatgagaggaatattggactgtacatttacaatcatagacaatgtatattacgaagagagttacttttgcgggaggaactgataactgctcaaggacttgtaacagcacacgttcgccaatttgcaccttggctggtgattccacgtacaataggagtcgttcgtacagtacttgtaggatcaacaggtctcgggtgcagggttaatctccttttatgactattaaccttgaaatgtgagacacatggctgttatatcaaggagaaaagaactcctatcattaacagggggggggcatgtagatatccggggtgttaaccgtgtaggtttggaccaaacaccgacagacgtttgacagtgacaggtggggtaatggctggaaatctcgaagcggaagtttcttatctgttggatctactgctacactttgttgaaacaataccagctgtcttttcattactttggcacttttttttttttttttttgaagtcatagtcgagggatgcgattcgaacctgctgcacagcaaagtattgcgctataggtacaaaagtaatgtcaaaacaatcgcctgatccagcttgacgtataccgcctacagtatgtacagacatatacctcccgttactgtaagattggtacggctgtagcattttagcgccgtcgtgtttttagcgtcgttcgtattttggcgtgagtttagcgtgattttagtatggtgtctatttctcggttgtccaaagcatcagcagaagagaagtcatacaagtcttcggtttcaagccttcggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatcctattcgggcaagaccaacaggcccgagctggtgacatttaccccagctggattgcaccagatcttggcattgggtggattcaaacaagggtatgtcatagacaggaggacgtgtaacagtccgtgatgatactggtatgggatatagttagcatcgtcgagaatagatgggacttgaaattcccaaaaccgaacggttctcggcactttttccaacaacttttggtatcataaagtatctgtttggtacggtatcaccttctcaccgggaaataccagctcgtttccccatcgtgtgttgtatgccactccacacttggctgacttgccaagcaaccttgtgagaagaaaaaggaaaaaaacggagcgaaatgaagagtcggaaaattgttggtttttttttgtttttagaacccgaaagtagccgcagaggggaaccacagggtcgttgaacagttgcttttaggatgtactgatcgggaaaggctgtccgaaggcaagagtgtgtagtatagcagaggcgatactggaatggatcagtaggtattccacctgtggaccaagtacctccggcttttgatacagcactatagcagattttgagctgacaccccacaaatgtcagtgtcaagaatgca

SEQ ID NO: 6 - DNA, 6789 bp, Artificial Sequence
  >pUC_JEN6_PUT_for Ligation of pUCLys2_DK2* into pUC_JEN3_P_T*
gatcctattcgggcaagaccaacaggcccgagctggtgacatttaccccagctggattgcaccagatcttggcattgggtggattcaaacaagggtatgtcatagacaggaggacgtgtaacagtccgtgatgatactggtatgggatatagttagcatcgtcgagaatagatgggacttgaaattcccaaaaccgaacggttctcggcactttttccaacaacttttggtatcataaagtatctgtttggtacggtatcaccttctcaccgggaaataccagctcgtttccccatcgtgtgttgtatgccactccacacttggctgacttgccaagcaaccttgtgagaagaaaaaggaaaaaaacggagcgaaatgaagagtcggaaaattgttggtttttttttgtttttagaacccgaaagtagccgcagaggggaaccacagggtcgttgaacagttgcttttaggatgtactgatcgggaaaggctgtccgaaggcaagagtgtgtagtatagcagaggcgatactggaatggatcagtaggtattccacctgtggaccaagtacctccggcttttgatacagcactatagcagattttgagctgacaccccacaaatgtcagtgtcaagaatgcagtatgaagaagcatcgtgtgagtttgccagtatctccatacaccagccaggatcgtgagtcccaccggataatccgtgccaagacgaaaacctttctgcactagcagtctctggcgtctcacacgtccattttctccgccgcgcatggaaaccacgctaatgaaactctcaccttgttgggaaagggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcccgtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtaccgagcgactgacaatcattcttccgtaccacagatacgagtcccggttcgtgggaattattaatgacgaggtccacgtgtggagattcaattggggatacatcaaggacgtctttgatcacaagcgacatggtaaggaggagtatgacttttcagaggtggagaagcacggtagttgatgaaatgagaggaatattggactgtacatttacaatcatagacaatgtatattacgaagagagttacttttgcgggaggaactgataactgctcaaggacttgtaacagcacacgttcgccaatttgcaccttggctggtgattccacgtacaataggagtcgttcgtacagtacttgtaggatcaacaggtctcgggtgcagggttaatctccttttatgactattaaccttgaaatgtgagacacatggctgttatatcaaggagaaaagaactcctatcattaacagggggggggcatgtagatatccggggtgttaaccgtgtaggtttggaccaaacaccgacagacgtttgacagtgacaggtggggtaatggctggaaatctcgaagcggaagtttcttatctgttggatctactgctacactttgttgaaacaataccagctgtcttttcattactttggcacttttttttttttttttttgaagtcatagtcgagggatgcgattcgaacctgctgcacagcaaagtattgcgctataggtacaaaagtaatgtcaaaacaatcgcctgatccagcttgacgtataccgcctacagtatgtacagacatatacctcccgttactgtaagattggtacggctgtagcattttagcgccgtcgtgtttttagcgtcgttcgtattttggcgtgagtttagcgtgattttagtatggtgtctatttctcggttgtccaaagcatcagcagaagagaagtcatacaagtcttcggtttcaagccttcggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagta

SEQ ID NO: 7 - DNA, 4480 bp, Artificial Sequence
  >JEN4_DK pUC_J4_PUT_for 446 to 4925
cgccgagtcgtaaagaagagatacctgctgcgcgagctttgccaccagagagtttcgtgagccactcagcagcgatctctgccagaaacattcctgtgcttgggccaacatgagatctctaacacagcaaatggtgtcatattcgagtcctcgcatgttgagactttccaagtctggcaagccgttctcaatgagaatctggaagcatcctgctgccatttgaaagtaattgaatgacatctttagaccgtctgcgtcctgccttccctcattgacgcccatctgactgtacagcgagcccaggttgtagagaagattgcatcgctcgaaatgaaggctttgactgatgacgggggcggcattggcggtgtaggccagagttccgtaccacatgaactccaggctatcgcatccggctggaaacttggtagtccagtatgtcaattgggcgtagtacttggtgaggtggtcgagatagctggcgtttggttgcgggttgagaatgttgttacgcaggtcgttggctgttttcaggtcggctgtaaacttgtctcgaggttggttcagctgcttctcgatggtgtcgcccagtcctgtgattagatcgacagcttgtgtctcccgaaacgggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatccgacagattgagcgagtagacaggtctatatatacgcctggatatcaaaagtcgtgtttaatcattaaatggcgagatcatgacccctcaggcgtgaactgcttttccgccctttagtctagcggtccaaccacagtcttattggtgaaacattgtggccatgtgtagtcccggaatggatagagacagtattggtctgttattgtcccatacgagagccaagaagatactgtggagtcttatataatgacatgctctcaagtatctgcagccttgcgcaacgtctcactcctcaacgacagcttctccaacttcgtcagtccaaattagtacgttgagtccccgcctccggtccgcttctacacgagtatatacgtagttatatacatactcgtagctcttgtattataaatcgactgtcggaaaatctttttagaccagagataggattagaaatgtagacgtcaagtatgtactagcgatgatagtcatcgagcccaacctctggcgcactttgactgggggccaaatgaacagtagcctgatagcacacacgtagggggaggattgacgtcttaatagatcacaagtgcatcatctgctgccgcaatcgttcctcattccaccattcatatagtcaataagtatacaaacgtattcgcttagaacttctccacttccccactaaaaagtactaaacgacccggatcgccacttccggaaggacccacctacttgtgctgtatctacccgagcgcaggagcaccccggagcagaggccttcaccagccctgttgctccatcagctgttcaaaatgcacaataaataattacttaactaatcgactcctaatggccacccttggcggcctgctccttaaccttcaactcacggtatttatcctgcgcggcacgttccttcttgacgagccagtcaacctcctcctgcagagccgcgcttcgctgctcaatggcaat

SEQ ID NO: 8 - DNA, 7217 bp, Artificial Sequence
  >pUC_JEN4_PUT_for Ligation of pUCLys2_DK2* into pUC_J4_PT*
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgtaccttctgagcataccctttcctgtcccctccttgatggcattaagagcagccttgagacgtgcaatctcctcgccatatctgcatccatccagtgcgtcacgtgccattcggtaatgtgcgaccgctagaaagtgctgtcgtttgcacgtcatgtgatgcacccattcgcttcggacctgcgggctcttttgggcccaggcgagcgccgagtcgtaaagaagagatacctgctgcgcgagctttgccaccagagagtttcgtgagccactcagcagcgatctctgccagaaacattcctgtgcttgggccaacatgagatctctaacacagcaaatggtgtcatattcgagtcctcgcatgttgagactttccaagtctggcaagccgttctcaatgagaatctggaagcatcctgctgccatttgaaagtaattgaatgacatctttagaccgtctgcgtcctgccttccctcattgacgcccatctgactgtacagcgagcccaggttgtagagaagattgcatcgctcgaaatgaaggctttgactgatgacgggggcggcattggcggtgtaggccagagttccgtaccacatgaactccaggctatcgcatccggctggaaacttggtagtccagtatgtcaattgggcgtagtacttggtgaggtggtcgagatagctggcgtttggttgcgggttgagaatgttgttacgcaggtcgttggctgttttcaggtcggctgtaaacttgtctcgaggttggttcagctgcttctcgatggtgtcgcccagtcctgtgattagatcgacagcttgtgtctcccgaaacgggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatccgacagattgagcgagtagacaggtctatatatacgcctggatatcaaaagtcgtgtttaatcattaaatggcgagatcatgacccctcaggcgtgaactgcttttccgccctttagtctagcggtccaaccacagtcttattggtgaaacattgtggccatgtgtagtcccggaatggatagagacagtattggtctgttattgtcccatacgagagccaagaagatactgtggagtcttatataatgacatgctctcaagtatctgcagccttgcgcaacgtctcactcctcaacgacagcttctccaacttcgtcagtccaaattagtacgttgagtccccgcctccggtccgcttctacacgagtatatacgtagttatatacatactcgtagctcttgtattataaatcgactgtcggaaaatctttttagaccagagataggattagaaatgtagacgtcaagtatgtactagcgatgatagtcatcgagcccaacctctggcgcactttgactgggggccaaatgaacagtagcctgatagcacacacgtagggggaggattgacgtcttaatagatcacaagtgcatcatctgctgccgcaatcgttcctcattccaccattcatatagtcaataagtatacaaacgtattcgcttagaacttctccacttccccactaaaaagtactaaacgacccggatcgccacttccggaaggacccacctacttgtgctgtatctacccgagcgcaggagcaccccggagcagaggccttcaccagccctgttgctccatcagctgttcaaaatgcacaataaataattacttaactaatcgactcctaatggccacccttggcggcctgctccttaaccttcaactcacggtatttatcctgcgcggcacgttccttcttgacgagccagtcaacctcctcctgcagagccgcgcttcgctgctcaatggcaatctcctccatgcgggctaactcggaaaacacggcaatctcgacctcaacagggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcccgtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 9 - DNA, 4668 bp, Artificial Sequence
  >JEN3_DK pUC_J3_PUT for 307 to 4974
cgcagaggcagtcaagaaacacaacattgctcgatacatccacgtgtctgcgttcaacgccgagattgactcgccctccgagttcaaccacaccaagggtctgggcgagcaggtcaccaaggacattgtgccctgggccaccattgtgcgaccggcccccatgtttggacgggaggacaagtggttcctggaccgaatggcccgatcgccctgtctggtgtccgccaacaagttccaggagacctccaaccccgtgcacgtgattgacgtggctgccgctctcgagcgaatctgcttcgacgactccaccgttgcccagacctttgagctgtacggcccccaaaagttcacccagaagcagatcattgacatggtttccgagaccctgcgaaaggaggtacgacacattgagctgcccaaggctctgtaccaggcttacaccaaggccacccaggccatctggtggcccacctactcccccgaccaggtcgagcgacagttcctgagccagaagattgatccttctgctaagaccttcaacgatctggacctgacccccatggagctgcccgatctcatgttcaagctgattcgaccctaccgagtcaacaccttccagcatgatgtgtcgcagctggagaacaaggagaagacttttgttcatattcttgactagtgtgtgagcgcaccgggcgaaatgaacacaagttcgtgattgagcgagcacactgcatcacaggacgacgactgtgggctgtcgaagacagtataaagtattagaagttgtattaaccagcagtgcagcgctgcaagtgtagtgggcaaagtgagatgctgtgtattgtagttgtaggtgacttacggaacgtctatccacggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatccggaatgctccagactaatcgtgtcgtctctatataccttctgaaagaggggtagggcaaatctagcgattttcatcgccaagcgacattacttctagtgtctttttcagcgagtatctgtatctgcaaatggggggtggagtgttgcgacttgagccgattcaggccaaagggaagaggaggaggggatgaggggattgcgaggccaggacgttgaaccgtttatagtaatgccttcgggaatcaacatcttcgtactccccagatggctcaacaagtgggggtcaagatggggtactgctatcgtagtgtggctgatagtcttccccacctgttagcgcgaagagacatgctgggggaaattgagccgagaaggggaccagagaagttggaagaaacgaagaatttttgcagctttcatcgtacttgttttccccgtactagttttcttcgtacttgtttcccttttggcatgttctcaactgtggccttttctggagatgttcccacagcaatatgtgaggcacaacggcctaggccctcaaacgcgccaatttcccggctactgcaacaatgtggggcatggagccaatcgtgggggtcacaattgaccagactgtcggttatcagactgctttgattgatacgtggggaaacaatccgttcagtggagttcagtggagagtgcaattgaactcagcagatgacagagcttagaagactccagcgctcggaaagatctgcatcctgtgtcaaaaagcagtttcaacttgccgtcatctccttcaaatgtcgtggggtacacacacatatatgcgctcggtattgtactgtgtgtactgtacttgaccgttccctgaacactagctacaaatagttcataccggtggtcggtaatgaaactactagagacccgtc

SEQ ID NO: 10 - DNA, 7404 bp, Artificial Sequence
  >pUC_J3_PUT for Ligation of pUCLys2_DK2* into pUC_J3_PT*
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttattggcagggagtacgagaccaagaacttcaactactacgacgtgcacgttgagggagcccgacgaatcgcagaggcagtcaagaaacacaacattgctcgatacatccacgtgtctgcgttcaacgccgagattgactcgccctccgagttcaaccacaccaagggtctgggcgagcaggtcaccaaggacattgtgccctgggccaccattgtgcgaccggcccccatgtttggacgggaggacaagtggttcctggaccgaatggcccgatcgccctgtctggtgtccgccaacaagttccaggagacctccaaccccgtgcacgtgattgacgtggctgccgctctcgagcgaatctgcttcgacgactccaccgttgcccagacctttgagctgtacggcccccaaaagttcacccagaagcagatcattgacatggtttccgagaccctgcgaaaggaggtacgacacattgagctgcccaaggctctgtaccaggcttacaccaaggccacccaggccatctggtggcccacctactcccccgaccaggtcgagcgacagttcctgagccagaagattgatccttctgctaagaccttcaacgatctggacctgacccccatggagctgcccgatctcatgttcaagctgattcgaccctaccgagtcaacaccttccagcatgatgtgtcgcagctggagaacaaggagaagacttttgttcatattcttgactagtgtgtgagcgcaccgggcgaaatgaacacaagttcgtgattgagcgagcacactgcatcacaggacgacgactgtgggctgtcgaagacagtataaagtattagaagttgtattaaccagcagtgcagcgctgcaagtgtagtgggcaaagtgagatgctgtgtattgtagttgtaggtgacttacggaacgtctatccacggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatccggaatgctccagactaatcgtgtcgtctctatataccttctgaaagaggggtagggcaaatctagcgattttcatcgccaagcgacattacttctagtgtctttttcagcgagtatctgtatctgcaaatggggggtggagtgttgcgacttgagccgattcaggccaaagggaagaggaggaggggatgaggggattgcgaggccaggacgttgaaccgtttatagtaatgccttcgggaatcaacatcttcgtactccccagatggctcaacaagtgggggtcaagatggggtactgctatcgtagtgtggctgatagtcttccccacctgttagcgcgaagagacatgctgggggaaattgagccgagaaggggaccagagaagttggaagaaacgaagaatttttgcagctttcatcgtacttgttttccccgtactagttttcttcgtacttgtttcccttttggcatgttctcaactgtggccttttctggagatgttcccacagcaatatgtgaggcacaacggcctaggccctcaaacgcgccaatttcccggctactgcaacaatgtggggcatggagccaatcgtgggggtcacaattgaccagactgtcggttatcagactgctttgattgatacgtggggaaacaatccgttcagtggagttcagtggagagtgcaattgaactcagcagatgacagagcttagaagactccagcgctcggaaagatctgcatcctgtgtcaaaaagcagtttcaacttgccgtcatctccttcaaatgtcgtggggtacacacacatatatgcgctcggtattgtactgtgtgtactgtacttgaccgttccctgaacactagctacaaatagttcataccggtggtcggtaatgaaactactagagacccgtcaaaacaatcaagtgcttctgaaaggacgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcccgtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 11 - DNA, 4204 bp, Artificial Sequence
  >JEN5_DK pUC_JEN5_PUT_for cut Van91I..601 to NdeI..4805
ttggctgcatttatcaagtgtctatcagtgcttgtatgaaaatccctcatgtgcgcactaattatcggtagttttgttcatgcacggcaaaacattagcccatgacaaaaaaaatgaacaaaaaagaggacgacaggactcgaacccgcagcctttggaggacgtcgttcagaagaaccgaaatccaatgcgctaccattacgccacgtcctcagattttcacgaactttttaaaaacttgccactactatcaaaaaactgattttttgaatttttttattgaaagccgataacccggcttactacaagtaggtatctagctgtgatgaatcactagaatcaaaaacacctcttgagtccactaaagagtcccggaggttggctgtagccacctacaagcccttctacgaacaatgtgtggaaacccccatagcccatgtccgaagctcagatcaactatctcttgctcctgctgtcgatgacgctctgtgaggccatgaccaactggcgtactatacgtattccacgctcatgaagaacaagctttgaatcaatttagtcattcttacgcggtacagaacggagaagaaggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatccttgccacgagacagtaagtgtatcagacagatgtaatgaatgcgattgcctcgtctaggtgaagttgatggttgtcaggtcgtatctcaggacaggcccgagggtatatatacatcctccagagactgaaactcttcataaaggttgcctcaaccccaccgtgcttcatatcaattacatttcgcattgccgcaactcggcacggatcaattaactccgtgtggggacaactctgtggaagagagacttcttagttccaggtgagtttccgagccaaacaaagaccagtcgtagtgacgtaatattgttgtgcagaaaagacatgttcactgcacgggaatacagaggtatcagtcagtctttgcagttttatatttcaggtttctggggttcaaattgacgataccgaggaaccgaaaaagcttgctgattgggaactgggagggccaaaactggtgagtgaagaagtagccgcaccaatttagatcgatgagtagttggaaatttgagaattggaaaaaaccgtcagaatcgccttggaatgaaaaatctgacagttttgttctagttaaaatcacatgactttgagtctagatcctcctgtgccaacattggctcaaagtttccgcccttgctcctttttgcccgactccaaaacaaaggcgcgtctaaatgaggtagccgtgggcgcctgatgcggtattttctccttacgcatctgtgcggtatttcacaccgca

SEQ ID NO: 12 - DNA, 6993 bp, Artificial Sequence
  >pUC_JEN5_PUT_for Ligation of pUCLys2_DK2* into puc jen 6 t p*
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttccatgggatatcgcatgcctgcagtagaccgaccatgagattgattggttactggcagattaaatcacccctcgtaatggcgcagcagctttaagacatacgatatgatacacaccaatggccgttgagagttacaacgtcgactggatacacagcccagtcatctgacagcgcaacggaagggggtgtgaacatggtggtacgcacaagcattagctgttgacttaaagaaggagcttcatgtgttattaatatctaccgatgggctgtattgaatatttgactgctcctttggctacttgtagtacagtactcattaccaggcaatacggtgatgcgtttaagcagtgctttggccagctcttggctgcatttatcaagtgtctatcagtgcttgtatgaaaatccctcatgtgcgcactaattatcggtagttttgttcatgcacggcaaaacattagcccatgacaaaaaaaatgaacaaaaaagaggacgacaggactcgaacccgcagcctttggaggacgtcgttcagaagaaccgaaatccaatgcgctaccattacgccacgtcctcagattttcacgaactttttaaaaacttgccactactatcaaaaaactgattttttgaatttttttattgaaagccgataacccggcttactacaagtaggtatctagctgtgatgaatcactagaatcaaaaacacctcttgagtccactaaagagtcccggaggttggctgtagccacctacaagcccttctacgaacaatgtgtggaaacccccatagcccatgtccgaagctcagatcaactatctcttgctcctgctgtcgatgacgctctgtgaggccatgaccaactggcgtactatacgtattccacgctcatgaagaacaagctttgaatcaatttagtcattcttacgcggtacagaacggagaagaaggatccaggaggacgagaaggtcaacaagggtgaggcctggagagagttctacaagggtccccgggatcggctgtacagaactggagatccaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagaattctggtggtattgtgactggggatgtagttgagaataagtcatacacaagtcagctttcttcaagcctcatataagtataagtagttcaatcgtattagctacttgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacaggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagatccttgccacgagacagtaagtgtatcagacagatgtaatgaatgcgattgcctcgtctaggtgaagttgatggttgtcaggtcgtatctcaggacaggcccgagggtatatatacatcctccagagactgaaactcttcataaaggttgcctcaaccccaccgtgcttcatatcaattacatttcgcattgccgcaactcggcacggatcaattaactccgtgtggggacaactctgtggaagagagacttcttagttccaggtgagtttccgagccaaacaaagaccagtcgtagtgacgtaatattgttgtgcagaaaagacatgttcactgcacgggaatacagaggtatcagtcagtctttgcagttttatatttcaggtttctggggttcaaattgacgataccgaggaaccgaaaaagcttgctgattgggaactgggagggccaaaactggtgagtgaagaagtagccgcaccaatttagatcgatgagtagttggaaatttgagaattggaaaaaaccgtcagaatcgccttggaatgaaaaatctgacagttttgttctagttaaaatcacatgactttgagtctagatcctcctgtgccaacattggctcaaagtttccgcccttgctcctttttgcccgactccaaaacaaaggcgcgtctaaatgaggtagccgtgggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcccgtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 13 - DNA, 4685 bp, Artificial Sequence
  >MAE1-DK PUT pucbm21_mae_t_p_ub rev 2178 to 6862
aagcgggtcctcaagacaactttaatgtgtaaatgggtgggaaaaaaagaatagatattaataccaacatgcgataagcgcaactaatatcggatcgttgtttatcgacctttcggacttttgattggttcattttcttgttatgttcttcctctatttgtgtagtacaatcgcttagacactctttcggctatcagcccgggctgccgacagtgcattaatttttttaatttaatttttttaaaattttaattttactgaatatgcgaaaatgcacattgagagctgagggcggtggagaaagtaatgaggggaagttggttggggtagtttttagttgtgaccttagttgggcggcgatgggcggtggtttattttttcttcggtcacgtgactagtttgattttattcgatctattgggcttttgattggctgtgtctagagcatactggagctgccctgttgtcaagccccaccgttacgtgtggctcgggtttttttaaaattttcgaatcagactttttttgtgcgcacagttagagttggcagagatgagtggctagagagagccacttcgatacgagtgaaggtggcgttttgtggcgggttgcaaactgcgatgatgagggtcagagggggtttgttagtatcaaaatgatgagaaaaatttccagagacgattaatatgttttttgttggcactgaacatccgatttatcactctaccgcctgtcgtccgtatccctccctacatccggcaaggttggtccaaggtttagcaatgtggtggctaacccccactcgccacaagtccgttgcccccttgggcaaaaccctagtccacatccgctctgattgcgtctaattagcaaagtattaaagtgcagccaccgccacagttttaaacaacacctaccatcgatttcgagcagggatctactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctcctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccagaattctactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctggatctccagttctgtacagccgatcccggggacccttgtagaactctctccaggcctcacccttgttgaccttctcgtcctcctggatcccatgcactgtaggcgacatctcataatctatcataatggtggtattaatgtctatgtgtataaatatctaattaatgaatgcaagttaattcttggatctgtttttccactgaaagtaaagatactggacgccgacaaagcagtccagagatgtctggatgactgagcacatgatgaactgcgacgagatctctgcttttccagaaaggaagtacgccagcttggccaagtctcctcctagccatgataccagcagcgacagtcggaacccatcgacactctgtcgctggtggtttttgatgatttggggtagaggcagaactgcctcgatcagcagcccaatggctccaatggaatccacgtagatggggtaggagccaaacagaaactgcaaaatgactagccacacggaaaacttgacaaggaaggaccagtacaggtcgtcgttgctccagttccagaagttgaacggtctcgaggacgacgtgtgcaggtcagggtacagagacttgcctctggacttgaacgctgaggaaacggatcgagcaaactgctgtctgtgctccgaccggtaggtttccggcgagatggcgtgcttcacggctgcgtacgtggccttcaggggtcccaggagtcccagaaactccagcactcgttcggtgtctgatcccggaggagtggtcttttcagctgaggcagacgcggcggtgctcatgtttccgagctcaatgtcgcgttcatcggctcgaggtcggtactgcagagcgtacttgagcagaaacaggtgcatgatgatcatgacgattgactggaccagtagagagagctcaaacggtcgtccgaaccagaacatgacacgtagaatcgacgacaccagc

SEQ ID NO: 14 - DNA, 6897 bp, Artificial Sequence
  >pUCMAE1_PUT_for Ligation of inverted pUCLys2_DK2* into pucbm21_maet_p*
gcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcccgtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgtgggtctcgggttatttctgtacttaactccgcattgtggcgtgggggggtcagccacaagcgggtcctcaagacaactttaatgtgtaaatgggtgggaaaaaaagaatagatattaataccaacatgcgataagcgcaactaatatcggatcgttgtttatcgacctttcggacttttgattggttcattttcttgttatgttcttcctctatttgtgtagtacaatcgcttagacactctttcggctatcagcccgggctgccgacagtgcattaatttttttaatttaatttttttaaaattttaattttactgaatatgcgaaaatgcacattgagagctgagggcggtggagaaagtaatgaggggaagttggttggggtagtttttagttgtgaccttagttgggcggcgatgggcggtggtttattttttcttcggtcacgtgactagtttgattttattcgatctattgggcttttgattggctgtgtctagagcatactggagctgccctgttgtcaagccccaccgttacgtgtggctcgggtttttttaaaattttcgaatcagactttttttgtgcgcacagttagagttggcagagatgagtggctagagagagccacttcgatacgagtgaaggtggcgttttgtggcgggttgcaaactgcgatgatgagggtcagagggggtttgttagtatcaaaatgatgagaaaaatttccagagacgattaatatgttttttgttggcactgaacatccgatttatcactctaccgcctgtcgtccgtatccctccctacatccggcaaggttggtccaaggtttagcaatgtggtggctaacccccactcgccacaagtccgttgcccccttgggcaaaaccctagtccacatccgctctgattgcgtctaattagcaaagtattaaagtgcagccaccgccacagttttaaacaacacctaccatcgatttcgagcagggatctactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctcctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccagaattctactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctggatctccagttctgtacagccgatcccggggacccttgtagaactctctccaggcctcacccttgttgaccttctcgtcctcctggatcccatgcactgtaggcgacatctcataatctatcataatggtggtattaatgtctatgtgtataaatatctaattaatgaatgcaagttaattcttggatctgtttttccactgaaagtaaagatactggacgccgacaaagcagtccagagatgtctggatgactgagcacatgatgaactgcgacgagatctctgcttttccagaaaggaagtacgccagcttggccaagtctcctcctagccatgataccagcagcgacagtcggaacccatcgacactctgtcgctggtggtttttgatgatttggggtagaggcagaactgcctcgatcagcagcccaatggctccaatggaatccacgtagatggggtaggagccaaacagaaactgcaaaatgactagccacacggaaaacttgacaaggaaggaccagtacaggtcgtcgttgctccagttccagaagttgaacggtctcgaggacgacgtgtgcaggtcagggtacagagacttgcctctggacttgaacgctgaggaaacggatcgagcaaactgctgtctgtgctccgaccggtaggtttccggcgagatggcgtgcttcacggctgcgtacgtggccttcaggggtcccaggagtcccagaaactccagcactcgttcggtgtctgatcccggaggagtggtcttttcagctgaggcagacgcggcggtgctcatgtttccgagctcaatgtcgcgttcatcggctcgaggtcggtactgcagagcgtacttgagcagaaacaggtgcatgatgatcatgacgattgactggaccagtagagagagctcaaacggtcgtccgaaccagaacatgacacgtagaatcgacgacaccagcatgatgccacagacgtcaatactgaagcccgcggc